(19) United States
(12) Patent Application Publication
(10) Pub. No.: US 2007/0274907 A1
(43) Pub. Date: Nov. 29, 2007

(54) Magnetically Targetable Mitomycin C Compositions and Methods of Their Use

(76) Inventors: Yuhua Li, Newark, DE (US); Caryn Peterson, Encinitas, CA (US); Scott R. Rudge, Boulder, CO (US); Suzanne Stevens, Longmont, CO (US); Gilles H. Tapolsky, Boulder, CO (US)

Correspondence Address: THE MCCALLUM LAW FIRM, P. C., 685 BRIGGS STREET, PO BOX 929, ERIE, CO, 80516, US

(21) Appl. No.: 10/593,045

(22) PCT Filed: May 12, 2004
(86) PCT No.: PCT/US04/14890

§ 371 Date: Mar. 23, 2007

Related U.S. Application Data
(60) Provisional application No. 60/556,605, filed on Mar. 26, 2004.

(51) Int. Cl. A61K 51/00; A61K 49/10; A61K 9/14; A61K 31/407

(52) U.S. Cl. 424/1.11; 424/9.32; 424/489; 514/410

(57) ABSTRACT

The present embodiments are directed to compositions and methods of delivering the compositions that comprise mitomycin C and superior magnetically targetable carrier particles. As the compositions are magnetically targetable, their use allows the localization of mitomycin C to particular locations within a patient. This allows for an increase in the effective concentration or a decrease in systemic exposure of mitomycin C in a patient.

### BACKGROUND OF THE EMBODIMENTS

1. Field of the Invention

This invention relates to superior compositions, methods of making, and methods of delivering magnetically targetable carrier particles which contain mitomycin C.

2. Background of the Embodiments

Metallic carrier compositions used in the treatment of various disorders have been heretofore suggested and/or utilized (see, for example, U.S. Pat. Nos. 4,849,209, issued Jul. 18, 1989 to Lieberman et al. and 4,106,488, issued Aug. 15, 1978 to Gordon), and have included such compositions that are guided or controlled in a body in response to external application of a magnetic field (see, for example, U.S. Pat. Nos. 4,501,726, (issued Feb. 26, 1985 to Schroder et al.), 4,652,257 (issued Mar. 24, 1987 to Chang), and 4,690,130 (issued Sep. 1, 1987 to Mirell)). Such compositions have not always proven practical and/or entirely effective. For example, such compositions may lack adequate capacity for carriage of the desired biologically active substance to the treatment site, have less than desirable magnetic susceptibility, and/or be difficult to manufacture, store and/or use. As proof, not a single of these previous attempts have reached commercial use.

One such known composition, deliverable by way of intravascular injection, includes microspheres made up of a ferromagnetic component covered with a biocompatible polymer (albumin, gelatin, and polysaccharides) which also contains a drug (Driscol C. F. et al. Prog. Am. Assoc. Cancer Res., 1980, p. 261).

It is possible to produce albumin microspheres up to 3.0 μm in size containing a magnetic material (magnetite Fe₃O₄) and the anti-tumoral antibiotic doxorubicin (Widder K. et al. J. Pharm. Sci., 68:79-82 1979). Such microspheres are produced through thermal and/or chemical denaturation of albumin in an emulsion (water in oil), with the water phase containing a magnetite suspension in a medicinal solution. A similar technique has been used to produce magnetically controlled, or guided, microcapsules covered with ethylcellulose containing the antibiotic mitomycin-C (Fujimoto S. et al., Cancer, 56: 2404-2410, 1985).

Another method is to produce magnetically controlled liposomes 200 nm to 800 nm in size carrying preparations that can dissolve atherosclerotic formations. This method is based on the ability of phospholipids to create closed membrane structures in the presence of water (Gregoriadis G., Ryman B. E., Biochem. J., 124:58, 1971).

The above compositions require extremely high flux density magnetic fields for their control, and are somewhat difficult to produce consistently, sterilize, and store on an industrial scale without changing their designated properties.

To overcome these shortcomings, a method for producing magnetically controlled dispersion has been suggested (See European Patent Office Publication No. 0 451 299 A1, by Kholodov L. E., Volkonsky V. A., Kolesnik N. F. et al.), using ferrocarbon particles as a ferromagnetic material. The ferrocarbon particles are produced by heating iron powder made up of particles 100 μm to 500 μm in size at temperatures of 800° C. to 1200° C. in an oxygen-containing atmosphere. The mixture is subsequently treated by carbon monoxide at 400° C. to 700° C. until carbon particles in an amount of about 10% to 90% by mass begin emerging on the surface. A biologically active substance is then adsorbed on the particles.

This method of manufacturing ferrocarbon particles is rather complicated and requires a considerable amount of energy. Because the ferromagnetic component is oxidized due to the synthesis of ferrocarbon particles at a high temperature in an oxygen containing atmosphere, magnetic susceptibility of the dispersion obtained is decreased by about one-half on the average, as compared with metallic iron. The typical upper limit of adsorption of a biologically active substance on such particles is about 2.0% to 2.5% of the mass of a ferromagnetic particle.

The magnetically controlled particle produced by the above method has a spherical ferromagnetic component with a threadlike carbon chain extending from it and is generally about 2.0 μm in size. The structure is believed to predetermine the relatively low adsorption capacity of the composites and also leads to breaking of the fragile thread-like chains of carbon from the ferromagnetic component during storage and transportation.

Mitomycin C (MMC) is a recognized FDA approved antineoplastic drug. It has a broad spectrum of clinical activity and a well-defined safety and efficacy profile. MMC is especially effective on fast growing tissues of all types and is actively cytotoxic against many cancers. It is commonly used alone or in combination with other agents in the treatment of Non Small Cell Lung Cancer (NSCLC) and cancers of the breast, stomach, pancreas, liver, and uterine cervix. Although not approved for the treatment of NSCLC, studies have documented that some NSCLC patients experience objective responses with MMC therapy. MMC is effective during the whole cell cycle, has well documented efficacy in pulmonary carcinoma (Wasserman et al., Cancer Chemotherapy, 3:399-419, 1975), and, unfortunately, has a serious side effect profile when delivered intravenously (Spain, Oncology 1993, 50 Suppl 1:35-50, 1993).

The delivery of MMC intravenously is limited by its systemic toxicity. In particular, adverse reactions that have been observed are Bone Marrow toxicity, Hemolytic Uremic syndrome, Renal toxicity, Pulmonary toxicity, and Cardiac toxicity. While all of these can be serious problems, the possibility of inducing Pulmonary toxicity would suggest to one of skill in the art to avoid the introduction of mitomycin C into the pulmonary artery.

Patients receiving systemic MMC experience a wide variety of undesirable adverse events, including myelosuppression, nausea, vomiting, fever, and anorexia. Other side-effects may include headache, blurring of vision, confusion, drowsiness, syncope, fatigue, edema, thrombophlebitis, hematemesis, diarrhea, pain, malaise, and asthenia. Bladder Fibrosis/contraction has been reported with intravesical administration.

Thus, there remains a need for an effective biocompatible composition that is capable of being transported magnetically, and which is relatively easy to manufacture, store and use. Furthermore, there remains a need to find a delivery method of MMC to target area that minimizes the side effects of this drug. The embodiments set forth below meet these needs and provide further advantages as well.

### SUMMARY OF THE EMBODIMENTS

Some aspects of the present invention are described in the following numbered paragraphs:

1. A magnetically targetable carrier composition comprising a composite particle of activated carbon and iron, wherein the carbon is randomly distributed throughout the particle volume, wherein each particle includes a ratio of weight of iron to activated carbon in the range of from about 95:5 to about 50:50, and wherein each particle includes a ratio of weight of mitomycin C to a combined carbon and iron weight in the range of from about 1:100 to about 20:100.

2. The magnetically targetable carrier composition of Paragraph 1, wherein the ratio of weight of mitomycin C to a combined carbon and iron weight in the range of from about 1:100 to about 10:100.

3. The magnetically targetable carrier composition of Paragraph 1, wherein the ratio of weight of mitomycin C to a combined carbon and iron weight is about 5:100.

4. The magnetically targetable carrier composition of Paragraph 1, wherein the mean size of the particles is less than 5 μm.

5. The magnetically targetable carrier composition of Paragraph 1, wherein the mean size of the particles in the magnetic composition is between approximately 0.1 μm to approximately 20 μm.

6. The magnetically targetable carrier composition of Paragraph 1, wherein the mean size of the particle is from between about 0.5 μm to about 5 μm.

7. The magnetically targetable carrier composition of Paragraph 1, wherein a major dimension of about 95% of particles is between about 0.5 μm and about 5 μm.

8. The magnetically targetable carrier composition of Paragraph 1, wherein the activated carbon is selected from the group consisting of Norit A, B, E, and K, and chemically modified versions and combinations thereof.

9. The magnetically targetable carrier composition of Paragraph 1, wherein the activated carbon is a type KB carbon.

10. The magnetically targetable carrier composition of Paragraph 8 or Paragraph 9, wherein said weight ratio of iron to activated carbon is from about 85:15 to about 60:40.

11. The magnetically targetable carrier composition of Paragraph 8 or Paragraph 9, wherein the ratio of iron:carbon is 86:14 to 64:36.

12. The magnetically targetable carrier composition of Paragraph 8 or Paragraph 9, wherein the ratio of iron:carbon is 65%:35%.

13. The magnetically targetable carrier composition of Paragraphs 1, 8 or 9, wherein the iron contains about less than 5% iron oxide.

14. The magnetically targetable carrier composition of Paragraph 8 or Paragraph 9, wherein the mean diameter of the particles is between about 0.6 and about 3.5 microns.

15. The magnetically targetable carrier composition of Paragraph 1, wherein said particles have an adsorption capacity for an additional biologically active substance of up to 20% of the mass of the particle.

16. The magnetically targetable carrier composition of Paragraph 1, wherein composite particles have a therapeutically effective amount of an additional biologically active substance adsorbed thereon, the biologically active substance being different from mitomycin C, and said carbon is activated carbon.

17. The magnetically targetable carrier composition of Paragraph 16, wherein said additional biologically active substance is selected from the group consisting of a drug, a radioactive substance, genetic material or combination thereof.

18. The magnetically targetable carrier composition of Paragraph 16, wherein the one or more additional biologically active substances are selected from the group consisting of antibiotics, antifungals and an additional antineoplastic agents.

19. The magnetically targetable carrier composition of Paragraph 1, wherein said composite particles have a diagnostically effective amount of an additional biologically active substance absorbed thereon.

20. A formulation for a magnetically targetable carrier composition and mitomycin C, wherein the formulation comprises:

a magnetically targetable carrier composition of Paragraph 1;

a delivery vehicle.

21. The formulation of Paragraph 20, wherein the concentration of mitomycin C is between about 0.5 to about 1 mg/mL before adsorbtion to the magnetically targeted carrier.

22. The formulation of Paragraph 20, wherein the concentration of mitomycin C is about 0.75 mg/mL before adsorbtion to the magnetically targetable carrier.

23. The formulation of Paragraph 20, wherein an excipient is in the formulation.

24. The formulation of Paragraph 23, wherein the excipient is mannitol.

25. The formulation of Paragraph 23, wherein the concentration of the excipient is about 5 to about 10% of the weight of the final preparation.

26. The formulation of Paragraph 25, wherein the concentration of excipient is about 6.7%.

27. The formulation of Paragraph 20, wherein the delivery vehicle comprises:

a salt

a polymer; and

a solvent.

28. The formulation of Paragraph 27, wherein the sugar is maimitol.

29. The formulation of Paragraph 27, wherein the concentration of mannitol is 100 mg/mL.

30. The formulation of Paragraph 27, wherein the polymer is carboxymethylcellulose.

31. The formulation of Paragraph 27, wherein the concentration of carboxymethylcellulose is 3 mg/mL.

32. The formulation of Paragraph 27, wherein the delivery vehicle comprises mannitol, carboxymethylcellulose, and water.

33. The formulation of Paragraph 27, wherein the delivery vehicle comprises about 100 mg mannitol, about 3 mg carboxymethylcellulose, and about 897 mg water.

34. The formulation of Paragraph 27, wherein the delivery vehicle comprises a solution with a viscosity of about 14±2 cP when measured at 40 C and 60 rpm in a Brookfield viscometer.

The present embodiments are directed to compositions and methods of delivering the compositions which comprise mitomycin C (MMC) and superior magnetically targetable carriers. As the compositions are magnetically targetable, their use allows the localization of mitomycin C to particular locations within a patient. This allows for an increase in the effective concentration or a decrease in systemic exposure of mitomycin C in a patient.

In one embodiment, a magnetically targetable carrier composition comprising composite particles of activated carbon and iron is contemplated. In this embodiment, the particle is characterized as having carbon randomly distributed throughout the particle volume, having a ratio of weight of iron to activated carbon in the range of from about 95:5 to about 50:50, and having a ratio of weight of mitomycin C to a combined carbon and iron weight in the range of from about 1:100 to about 20:100.

In another embodiment, a formulation for a magnetically targetable carrier and mitomycin C is contemplated. The formulation comprises a magnetically targetable carrier, an amount of mitomycin C, wherein the mitomycin C is predominately associated with the magnetically targetable carrier, and a delivery vehicle.

Another embodiment includes a kit for administering a biologically active substance to an in vivo site in a patient comprising: a first receptacle comprising a unit dose of dry ferrocarbon particles between 0.1 μm and 5 μm in major dimension, each particle including a ratio of iron to activated carbon in the range from about 95:5 to about 50:50, said carbon and iron forming a composite; one or more dry excipients in an amount that enhances adsorption of said biologically active substance to said particles when in solution; and an amount of mitomycin C adsorbed onto said particles.

Further embodiments include a method for delivering an antineoplastic drug to a tissue in a patient, said method comprising: administering a composition comprising composite particles of activated carbon and iron, wherein the carbon is randomly distributed throughout the particle volume, wherein each particle includes a weight ratio of iron to activated carbon in the range of from about 95:5 to about 50:50, and wherein each particle includes a ratio weight of mitomycin C to a combined carbon and iron weight in the range of from about 1:100 to about 20:100 to a patient; and localizing the distribution of the composition in the patient through the use of a magnetic field for a period of time long enough to deliver a drug to a tissue in need of treatment; the above method, wherein the tissue to be treated is selected from the group consisting of lung, liver, and pancreas; the above method, wherein the particles are introduced by a method selected from the group consisting of injection, infusion, implantation, and ingestion; the above method, wherein the composition of magnetically targetable carrier and MMC is administered to a tissue through an artery, preferably the pulmonary, bronchial, hepatic or femoral arteries; the above method, wherein the concentration of mitomycin C in circulation is reduced owing to the attachment of the MMC to the magnetically targeted carrier; and the above method, wherein the magnetically targetable carrier is administered intra-arterially.

Other embodiments include the above method, wherein the composition that is administered to the patient further comprises a delivery vehicle; the above method, wherein the weight ratio of mitomycin C to a combined weight of carbon and iron is about 5:100; the above method, further comprising the introduction of an embolic agent; the above method, wherein the composition is administered in unit dose form; the above method, wherein the wherein the activated carbon is selected from the group consisting of A, B, E, K, and especially type KB, and chemically modified versions and combinations thereof; and the above method, wherein the composition has a ratio of weight of mitomycin C to combined weight of iron and carbon of about 5:100.

Other embodiments include a method of inhibiting a cancer, said method comprising: administering a composition of magnetically targetable particles comprising activated carbon, mitomycin C, and metallic iron to a tissue in need of inhibition of a cancer; and localizing said composition at a cancer through the use of a magnetic field; and the above method wherein the cancer is a non small cell lung cancer, pancreatic cancer, liver cancer, sarcoma, stomach cancer, breast cancer or uterine cancer.

Another embodiment includes a method of inhibiting a cancer, said method comprising: administering a composition of magnetically targetable particles comprising activated carbon, mitomycin C, and metallic iron to a tissue in need of inhibition of a cancer; and localizing the magnetically targetable particles to a cancer in a patient by using a magnetic field; wherein an amount of mitomycin C in the composition is not sufficient to adequately treat a cancer if the amount of mitomycin C were distributed throughout the blood supply, but the amount is sufficient if concentrated at the cancer by a magnetic field.

Yet another embodiment includes a method for delivering an anti-scarring agent to a tissue in a patient, said method comprising: administering a composition comprising composite particles of activated carbon and iron, wherein the carbon is randomly distributed throughout the particle volume, wherein each particle includes a weight ratio of iron to activated carbon in the range of from about 95:5 to about 50:50, and wherein each particle includes a ratio weight of mitomycin C to a combined carbon and iron weight in the range of from about 1:100 to about 20:100 to a patient; and localizing the distribution of the composition in the patient through the use of a magnetic field for a period of time long enough to deliver a drug to a tissue in need of treatment.

Other embodiments include a method of delivering mitomycin C to a desired location in a patient, said method comprising: administering a composition comprising composite particles of activated carbon and iron, wherein the carbon is randomly distributed throughout the particle volume, wherein each particle includes a weight ratio of iron to activated carbon in the range of from about 95:5 to about 50:50, and wherein each particle includes a ratio weight of mitomycin C to a combined carbon and iron weight in the range of from about 1:99 to about 20:80 to a patient; and localizing the distribution of the composition in the patient through the use of a magnetic field at a desired location in a patient; and the above method, wherein the activated carbon of the composition is selected from the group consisting of: A, B, E, K, and KB, and chemically modified versions and combinations thereof.

Yet other embodiments include a method of reducing the systemic exposure of mitomycin C in a patient, said method comprising: administering a composition comprising composite particles of activated carbon and iron, wherein the carbon is randomly distributed throughout the particle volume, wherein each particle includes a weight ratio of iron to activated carbon in the range of from about 95:5 to about 50:50, and wherein each particle includes a ratio weight of mitomycin C to a combined carbon and iron weight in the range of from about 1:100 to about 20:100 to a patient; and reducing the systemic exposure of mitomycin C to the relatively healthy tissues in a patient by localizing the distribution of the composition in the patient through the use of a magnetic field to a location in a patient; in one embodiment, the patient is in need of treatment with mitomycin C; and in another embodiment, the patient is in need of treatment for a cancer.

Other embodiments include a method of preparing a magnetically targetable carrier containing mitomycin C, said method comprising: preparing a magnetically targetable carrier that can adsorb mitomycin C; preparing an amount of mitomycin C; combining the mitomycin C and the magnetically targetable carrier; and letting the mitomycin C and the magnetically targetable carrier incubate together for about 5 to about 60 minutes; the above method, wherein the mitomycin C is in a solution when the mitomycin C and the magnetically targetable carrier are combined; the above method, wherein the incubation period is about 30 minutes; the above method, further comprising the step of removing the excess, unbound mitomycin C from the combination of mitomycin C and magnetically targetable carrier; the above method, further comprising the addition of other excipients to the suspension; the above method, further comprising lyophilizing the resulting formulation; the above method, further comprising placing the lyophilized formulation into a container and adding a gas to remove other gases from the remainder of the container; and optionally sealing the container; and the above method, wherein the magnetically targetable carrier comprises composite particles of activated carbon and iron, wherein the carbon is randomly distributed throughout the particle volume, wherein the carbon is selected from the group consisting of types A, B, E, K, and KB, wherein each particle includes a weight ratio of iron to activated carbon in the range of from about 95:5 to about 50:50, and wherein each particle includes a ratio weight of mitomycin C to a combined carbon and iron weight in the range of from about 1:100 to about 20:100.

### BRIEF DESCRIPTION OF THE DRAWINGS

FIG. 1 is a sectional illustration of a magnetically targetable carrier particle.

FIG. 2 is a flow-chart showing one embodiment for manufacturing a magnetically targetable carrier particle.

FIG. 3 is a flow-chart showing one embodiment for manufacturing a magnetically targetable carrier particle.

FIG. 4 is a graph showing particle size distribution for magnetically targetable carrier particles.

FIG. 5 is a graph the magnetic susceptibility and hysteresis for one lot of the magnetically targetable carrier particles.

FIG. 6 is a photo of a 1000× Magnification Electron Micrograph of magnetically targetable carrier particles.

FIG. 7 is a photo of a 2500× Magnification Electron Micrograph of magnetically targetable carrier particles.

FIG. 8 is a photo of a 4000× Magnification Electron Micrograph of magnetically targetable carrier particles.

FIG. 9 is a photo of a 10,000× Magnification Scanning Electron Micrograph of magnetically targetable carrier particles.

FIG. 10 is a photo of a 16,000× Magnification Scanning Electron Micrograph of magnetically targetable carrier particles.

FIG. 11 is a photo of a 100,000× Magnification Scanning Electron Micrograph of magnetically targetable carrier particles.

FIG. 12 is a graph showing the water vapor adsorption isotherm.

FIG. 13 is a flow-chart showing an embodiment of the magnetically targetable carrier-MMC particle for injection preparation.

FIG. 14 is a graph showing the concentration of mitomycin remaining in the binding supernatant and mitomycin stock solution versus time (N=3, Mean ±SD).

FIG. 15 is a graph showing mitomycin desorption in human plasma at different magnetically targetable carrier particle concentrations.

FIG. 16 is a graph showing cell viability of MMC.

FIG. 17 is a pretreatment selective angiogram showing the diameter of pulmonary vessels.

FIG. 18 is a pretreatment selective angiogram in which the left pulmonary artery has been catheterized and a branch has been selected for infusion.

FIG. 19 is a pretreatment selective angiogram in which the left pulmonary artery has been catheterized and a branch has been selected for infusion of the high dose of magnetically targetable carrier-MMC particle.

FIG. 20 is a post-treatment selective angiogram following the second infusion of the high dose of magnetically targetable carrier-MMC particle (total dose of 80 mg magnetically targetable carrier particle and 4 mg MMC). A magnet and its position on the skin surface are seen on the right side of the angiogram. Patchy loss of parenchymal enhancement is seen laterally with adjoining mild peripheral pruning.

FIG. 21 is a photo showing a pulmonary granuloma with intralesional magnetically targetable carrier particles in targeted lung lobe of swine 28 days following delivery of the high dose of magnetically targetable carrier-MMC particle containing 80 mg magnetically targetable carrier particle and 4 mg MMC.

FIG. 22 is a photo showing perivascular magnetically targetable carrier particles in targeted lung lobe of swine 28 days following magnetic targeted delivery of magnetically targetable carrier particles.

FIG. 23 is a photo showing magnetically targetable carrier particles in alveolar capillaries of targeted lung lobe of swine 28 days following magnetic targeted delivery of magnetically targetable carrier particles.

### DETAILED DESCRIPTION OF THE PREFERRED EMBODIMENTS

Regional therapy achieved through targeted drug delivery could potentially improve efficacy by increasing drug concentration in the tumor while limiting systemic drug concentrations that produce systemic toxicities. The use of magnetically targeted carrier (MTC) particles (or the equivalent term, magnetically targeted carrier, for short) for drug delivery aims to target drugs to specific sites with selective catheterization and direct application of a magnetic field to the desired site in order to achieve prolonged release of high, localized concentration of drug by retention of magnetically targetable carrier particles in the region of interest.

In one embodiment, magnetically targetable carrier particles are delivered intra-arterially to the desired site in the presence of an externally applied magnetic field that aids in magnetically targetable carrier particle localization and retention in the targeted site by extravasation of the magnetically targetable carrier particles into the surrounding tissue (Goodwin et al, J Magnetism and Magnetic Materials, 194:132-139, 1999). Clinical studies performed to date with another magnetically targeted investigational drug, magnetically targetable carrier-DOX particle, have shown that magnetically targetable carrier particles in conjunction with doxorubicin can be administered to patients with hepatocellular carcinoma (presented at the American Society of Clinical Oncology, 2001). These studies have also documented that the magnet is able to localize the drug to the liver tumor in a durable manner, and that as a result, no detectable systemic blood levels of doxorubicin are measured. The present disclosure further reveals that mitomycin C can also be associated with the magnetically targetable carrier particles in a beneficial manner and used to deliver relatively high doses of mitomycin to relatively localized areas in a patient in need of treatment.

The magnetically targetable carrier particles are magnetically controllable, or guided, carrier composition of this embodiment includes composite, volume-compounded ferrocarbon particles of about 0.1 μm to about 5.0 μm in average major dimension, and preferably between about 0.5 μm and about 5.0 μm, containing about 1.0% to about 95.0% by mass of carbon, for example, between about 10% and 60%. About 15% to about 40% is the preferred range of carbon having been found to exhibit characteristics useful in many applications.

The magnetically targetable carrier particles are produced by mechanically milling a mixture of iron and carbon powders, without application of external heat. The composite iron:carbon magnetically targetable carrier particles so obtained may then be placed in a solution of a biologically active substance to allow adsorption of the biologically active substance to the magnetically targetable carrier particles. The magnetically targetable carrier particles are separated for desired size and magnetic susceptibility characteristics. Separation of the magnetically targetable carrier particles can occur before or subsequent to exposure to the biologically active substance.

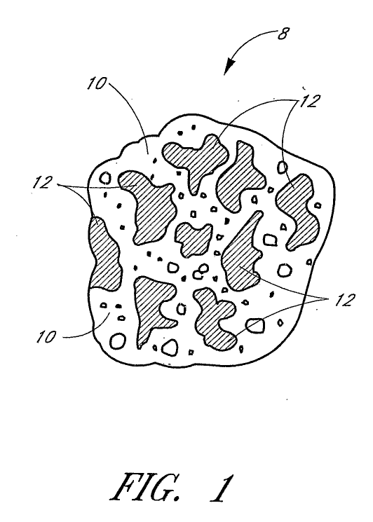

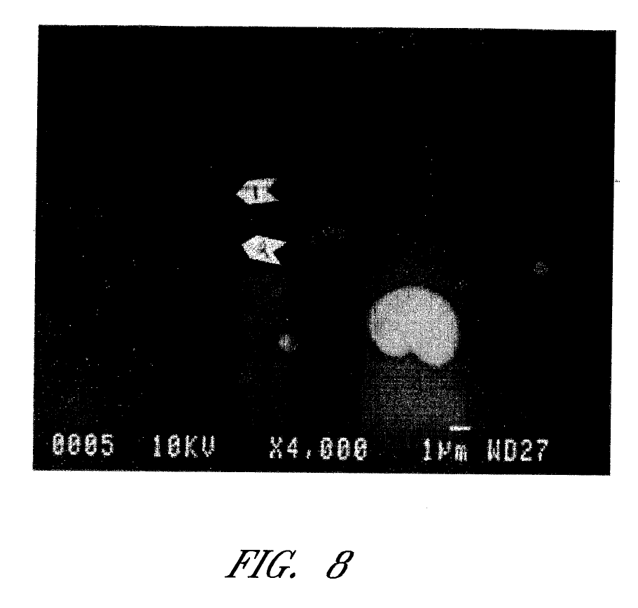

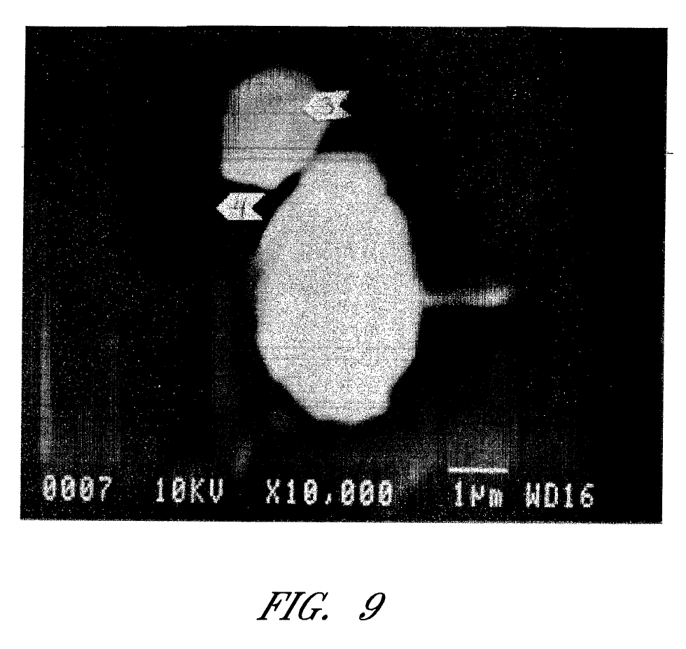

The iron:carbon magnetically targetable carrier particles manufactured by the methods of one embodiment are of a generally spherical shape with irregular detailed morphology, with the inclusions of carbon deposits being located throughout the whole volume of each magnetically targetable carrier particle (both at the surface and the interior of each magnetically targetable carrier particle), as will be shown below in greater detail in FIGS. 1 and 6-10. Thus, the carbon is substantially randomly distributed throughout the magnetically targetable carrier particle volume. The strong connection between the components is not broken during prolonged storage of the magnetically controlled composition, its transportation, storing, packing, and direct use. Chemical binding may take place between the iron and carbon, such as a trace interlayer of cementite (Fe₃C) or other iron carbide formed during the milling process.

The iron:carbon magnetically targetable carrier particles are useful as a carrier for delivering MMC and optionally one or more adsorbed biologically active substances to specific body sites under control of an external magnetic field. As used herein, the term “biologically active substance” includes substances useful for in vivo medical diagnosis and/or treatment. In another embodiment, mitomycin C is used to prevent any scarring of tissue in which the magnetically targetable carrier particles are located or associated tissue, and the second biologically active ingredient may be unrelated to cancer therapy. (See, for example, Wilkins M, Indar A, Wormald R., Intra-operative Mitomycin C for glaucoma surgery (Cochrane Review). In: The Cochrane Library, Issue 1, 2004. Chichester, UK: John Wiley & Sons, Ltd. for a discussion of mitomycin C in preventing scarring).

Generally, any useful diagnostic and/or therapeutic biologically active substance in addition to mitomycin C may be attached to the magnetically targetable carrier particles for guided delivery to a targeted site. The term “biologically active” also includes agents used for diagnostic purposes and having no apparent physiological, therapeutic effect. Bifunctional agents having both diagnostic and therapeutic properties are also contemplated. Biologically active substances that can be precipitated, adsorbed, or labeled onto the magnetically targetable carrier particles are, for example, but not limited to muscarinic receptor agonists and antagonists; anticholinesterase agents; catecholamines, sympathomimetic drugs, and andrenergic receptor antagonists; serotonin receptor agonists and antagonists; local and general anesthetics; anti-migraine agents such as ergotamine, caffeine, sumatriptan and the like; anti-epileptic agents; agents for the treatment of central nervous system degenerative disorders; opiod analgesics and antagonists; anti-inflammatory agents, including anti-asthmatic drugs; histamine and bradykinin antagonists, lipid-derived autocoids; nonsteroidal anti-inflammatory agents and anti-gout agents; anti-diuretics such as vassopressin peptides; inhibitors of the renin-angiotensin system such as angiotensin converting enzyme inhibitors; agents used in the treatment of myocardial ischemia, such as organic nitrates, Ca²⁺ channel antagonists, beta-adrenergic receptor antagonists, and antiplatelet/antithrombotic agents; anti-hypertensive agents such as diuretics, vasodilators, Ca²⁺ channel antagonists, beta-adrenergic receptor antagonists; cardiac glycosides such as digoxin, phosphodiesterase inhibitors; antiarrhythmic agents; anti-hyperlipoprotenimia agents; agents for the control of gastric acidity and treatment of peptic ulcers; agents affecting gastrointestinal water flux and motility; agents that cause contraction or relaxation of the uterus; anti-protozoal agents; anthelmintic agents; antimicrobial agents such as sulfonamides, quinolines, trimethoprim-sulfamethoxazole; beta-lactam antibiotics; aminoglycosides; tetracyclines; erythromycin and its derivatives; chloramphenicol, agents used in the chemotherapy of tuberculosis; Mycobacterium avium complex disease, and leprosy; anti-fungal agents; and anti-viral agents; anti-neoplastic agents such as alkylating agents, antimetabolites; natural products such as the vinca alkaloids, antibiotics (e.g., doxorubicin, bleomycin and the like); enzymes (e.g. L-asparaginase), biological response modifiers (such as interferon-alpha); platinum coordination compounds, anthracenedione and other miscellaneous agents; as well as hormones and antagonists (such as the estrogens, progestins, and the adrenocorticosteriods) and antibodies; immunomodulators including both immunosuppressive agents as well as immunostimulants; hematopoietic growth factors, anticoagulant, thrombolytic and antiplatelet agents; thyroid hormone, anti-thyroid agents, androgen receptor antagonists; adrenocortical steroids, insulin, oral hypoglycemic agents, agents affecting calcification and bone turnover as well as other therapeutic and diagnostic hormones, vitamins, minerals blood products biological response modifiers, diagnostic imaging agents, as well as paramagnetic and radioactive molecules or particles. Other biologically active substances may include, but are not limited to monoclonal or other antibodies, natural or synthetic genetic material, and prodrugs.

As used herein, the term “genetic material” refers generally to nucleotides and polynucleotides, including nucleic acids, RNA and DNA of either natural or synthetic origin, including recombinant, sense and antisense RNA and DNA. Types of genetic material may include, for example, genes carried on expression vectors, such as plasmids, phagemids, cosmids, yeast artificial chromosomes, and defective (helper) viruses, antisense nucleic acids, both single and double stranded RNA and DNA and analogs thereof. Also included are proteins, peptides and other molecules formed by the expression of genetic material.

In addition to delivering therapeutic amounts of MMC, the instance magnetically targetable carrier particles can be used for in vivo diagnostic imaging, the type of detection instrument available is a major factor in selecting a given radioisotope. The radioisotope chosen should have a type of decay that is detectable for a given type of instrument. Generally, gamma radiation is required. Still another important factor in selecting a radioisotope is that the half-life be long enough so that it is still detectable at the time of maximum uptake by the target, but short enough so that deleterious radiation with respect to the host is minimized. Selection of an appropriate radioisotope would be readily apparent to one having average skill in the art. Radioisotopes that may be employed include, but are not limited to ^{99m}Tc, ¹⁴²Pr, ¹⁶¹Tb, ¹⁸⁶Re, and ¹⁸⁸Re. Additionally, typical examples of other diagnostically useful compounds are metallic ions including, but not limited to ¹¹¹In, ⁹⁷Ru, ⁶⁷Ga, ⁶⁸Ga, ⁷²As, ⁸⁹Zr, and ²⁰¹TI. Furthermore, paramagnetic elements that are particularly useful in magnetic resonance imaging and electron spin resonance techniques include, but are not limited to ¹⁵⁷Gd, ⁵⁵Mn, ¹⁶²Dy, ⁵²Cr, and ⁵⁶Fe.

Biologically active substances such as radioisotopes are chemical agents or elements that emit alpha, beta, or gamma radiation and that are useful for diagnostic and/or therapeutic purposes. One factor used in selecting an appropriate radioisotope is that the half-life be long enough so that it is still detectable or therapeutic at the time of maximum uptake by the target, but short enough so that deleterious radiation with respect to the patient is minimized. Selection of an appropriate radioisotope would be readily apparent to one having ordinary skill in the art. Generally, alpha and beta radiation are considered useful for local therapy. Examples of useful agents include, but are not limited to ³²P, ⁵⁵Co, ⁵⁶Co, ⁵⁷Ni, ¹⁸⁶Re, ¹⁸⁸Re, ¹²³I, ¹²⁵I, ¹³¹I, ⁹⁰Y, ¹⁶⁶Ho, ¹⁵³Sm, ¹⁴³Pr, ¹⁴⁹Tb, ¹⁶¹Tb, ¹¹¹In, ⁷⁷Br, ²¹⁴Bi, ²¹³Bi, ²²⁴Ra, ²¹⁰Po, ^{195m}Pt, ¹⁶⁵Dy, ¹⁰⁹Pd, ^{117m}Sn, ^{123m}Te, ¹⁰³Pd, ¹⁷⁷Lu, and ²¹¹At. The radioisotope generally exists as a radical within a salt, with the notable exception of the Iodines. The useful diagnostic and therapeutic radioisotopes may be used alone or in combination.

As a general principle, the amount of any aqueous soluble biologically active substance or substances adsorbed can be increased by increasing the proportion of carbon in the magnetically targetable carrier particles up to a maximum of about 40% by mass of the magnetically targetable carrier particles without loss of utility of the magnetically targetable carrier particles in the therapeutic treatment regimens described in this application. In many cases it has been observed that an increase in the amount of adsorbed biologically active substance is approximately linear with the increase in carbon content. However, as carbon content increases, the susceptibility, or responsiveness, of magnetically targetable carrier particles to a magnetic field decreases, and thus conditions for their control in the body worsen (although adsorption capacity increases). Therefore, it is necessary to achieve a balance in the iron:carbon ratio to obtain improved therapeutic or diagnostic results. To increase the amount of agent given during a treatment regimen, a larger dose of magnetically targetable carrier particles can be administered to the patient, but the particles cannot be made more magnetic by increasing the dose. Appropriate ratios may be determined by one of skill in the art in light of the presently provided disclosure.

It has been determined that the useful range of iron:carbon ratio for magnetically targetable carrier particles intended for use in in vivo therapeutic treatments as described in the application is, as a general rule, from about 95:5 to about 50:50, for example about 85:15 to about 60:40. The maximum amount of the biologically active substance that can be adsorbed in the composite iron:carbon magnetically targetable carrier particles of any given concentration of carbon will also differ depending upon the chemical nature of the biologically active substance, and, in some cases, the type of carbon (e.g., activated carbon (AC)) used in the composition.

Adsorption of biologically active substances is affected by the quality of the solution they are dissolved in, that is, the concentration of the substance itself, the pH, ionic strength, tonicity, hydrophobic index, and temperature. Excipients may be added to alter some of these solution properties, either to aid in the solubilization of the biologically active substance, or to drive its adsorption to the particle. Solubilization can be increased through use of surfactants and solvents, adjustment to acidic or basic pH, as well as increases in temperature, or addition of energy, such as with sonication. Adsorption to the magnetic targetable carrier particles may be favored by increasing hydrophilicity, addition of sugars, salts or polymers, increase or decrease in temperature, neutral pH, and other factors dictated by the specific chemistry involved. As these two aims of solubility and adsorption to the particles are often in mutual opposition, the methods for achieving both are important to the design of the formulation, and would depend on the details of the specific chemistry and drug delivery goals for the product.

The suspension of magnetic targetable carriers must remain resistant to aggregation in the time prior to administration to a patient. Irreversible aggregation of the particles is undesirable as aggregates may block blood flow to the disease site, rapidly settle out of suspension, or cause uneven distribution of biologically active substance throughout the preparation or disease site. Polymers, sugars and surfactants are useful for inhibiting the aggregation of magnetic targetable carrier particles, but must be chosen as the correct type and in the correct concentrations in order to not interfere with the goals of solubilization and adsorption described previously.

Additionally, of particular interest, Example 1 illustrates the manufacture of magnetically targetable carrier particles useful for adsorption of mitomycin C to the magnetically targetable carrier particles. Using the methods of these embodiments, mitomycin C has been adsorbed onto magnetically targetable carrier particles having iron:carbon ratios from 64-86% to 36-14% respectively by weight. In one embodiment, the type of carbon used is type KB. Other biologically active substances may also, or additionally, be adsorbed using similar techniques that would be obvious to any person having average skill in the art, in light of the present disclosure. Each ratio of iron to carbon, and each ratio of biologically active ingredient to combined iron and carbon can be an invention in and of itself. Without the present disclosure, it can be difficult and unpredictable to determine the correct balance of carbon to iron. The relative amounts of each component, how the components are combined, the shapes of the components, and how large the components are can each influence the binding ability of the biologically active substance. In one embodiment, these magnetically targetable carrier particles have a minimum threshold of adsorption that is required in order for them to function in a patient, preferably a human patient. Not only is the binding of the biologically active substance important, but the desorption characteristics are also important. In one embodiment, the biologically active substance must attach to the magnetically targetable carrier particles strongly enough so as to allow delivery of the biologically active substance to a particular location. However, in one embodiment, they should also be able to release the biologically active substance in the patient so as to allow a greater degree of treatment. Furthermore, without the current disclosure, it can be difficult to determine the correct balance of mitomycin C (or other biologically active substance) to magnetically targetable carrier particle. The particular tests for determining adsorption and desorption properties, as well as particular results for mitomycin C are presented herein.

Because it is convenient to prepare and market the magnetically targetable carrier particles in a dry form, the excipients may be prepared in dry form, and one or more dry excipients are packaged together with a unit dose of the magnetically targetable carrier particles. A wide variety of excipients may be used, for example, to enhance precipitation or release of the biologically active substance, if present. A person having ordinary skill in the art readily can determine the types and amounts of appropriate dry excipients. The type and amount of appropriate dry excipients can readily be determined by any person having ordinary skill in the art. For instance, the excipients can be selected from a viscosity agent or a tonicifier, or both. Viscosity agents are, for example, biodegradable polymers such as carboxymethylcellulose, PVP, polyethylene glycol (PEG), and the like. Tonicifiers include sodium chloride, mannitol, dextrose, lactose, and other agents used to impart the same osmolarity to the reconstituted solution. Most preferably, the package or kit containing both the dry excipients and dry magnetic particles such as iron is formulated to be mixed with the liquid contents of a vial containing a unit dose of the biologically active substance. Liquid agents could be used as excipients just prior to use of the magnetically targetable carrier particles. Such liquid agents could be soybean oil, rapeseed oil, or an aqueous based polymer solution composed of the polymers as listed above. Also liquid solutions could be a tonicifier, such as Ringer's solution, 5% dextrose solution, physiological saline. As before a combination of liquid excipients and tonicifiers can be used. (See, for example, Kibbe, A H, Handbook of Pharmaceutical Excipients, American Pharmaceutical Association, Washington, D.C., 2000)). Upon mixture of the liquid containing the biologically active compound with the contents of the kit including the dry components (e.g., the dry iron particles and dry excipients), the biologically active compound attaches to the magnetically targetable carrier particles according to a protocol developed for each compound, thus forming a magnetically controllable composition containing a diagnostic and/or therapeutic amount of a biologically active compound attached to the magnetically targetable carrier particles and being suitable for ex vivo or in vivo therapeutic and/or diagnostic as well as ex vivo diagnostic use.

When a liquid kit is employed, the magnetically targetable carrier particles can be contained as one unit, for example, in a vial, while the aforementioned excipients are contained in another unit in the form of an aqueous solution. At the time of administration, the ferrocarbon particles (i.e., magnetically targetable carrier particles) are mixed with the contents of a vial containing a unit dose of the drug and sufficient amount of a biologically compatible aqueous solution, such as saline, as recommended by the drug manufacturer, to bring the drug to a pharmaceutically desirable concentration. Subsequently, the resulting magnetically targetable carrier particles having the biologically active substance adsorbed thereon, are mixed with yet another unit containing the excipients in aqueous solution. In manufacturing the two preparations, any suitable sterilization technique may be employed. For example, the ferrocarbon particles may be sterilized using gamma irradiation and the aqueous solution of excipients may be sterilized by autoclave.

A diagnostic or therapeutic amount of biologically active substance adsorbed to the magnetically targetable carrier particles will be determined by one skilled in the art as that amount necessary to effect diagnosis or treatment of a particular disease or condition, taking into account a variety of factors such as the patient's weight, age, and general health, the diagnostic or therapeutic properties of the drug, and the nature and severity of the disease.

A number of considerations are involved in determining the size of magnetically targetable carrier particles to be used for any specific therapeutic situation. The choice of particle size is determined in part by technological constraints inherent in producing the particles under 0.2 μm in size. In addition, for magnetically targetable carrier particles less than about 1.0 μm in size, the magnetic control in blood flow and the carrying capacity may be reduced. Relatively large particle sizes can tend to cause undesirable embolization of blood vessels during injection either mechanically or by facilitating clot formation by physiological mechanisms. The dispersion may coagulate, which makes injections more difficult, and the rate at which biologically active substances desorb from the magnetically targetable carrier particles in the targeted pathological zones may decrease. The method (such as is described below) of milling together a mixture of iron and carbon powders produces an approximately spherical form with a granular surface for the magnetically targetable carrier particles, and results in a particle population having an average major dimension of about 0.1 μm to about 5.0 μm.

Because the iron in the magnetically targetable carrier particles described in these embodiments is not in the form of an iron oxide, as is the case in certain previously disclosed magnetically controlled dispersions, the magnetic susceptibility, or responsiveness, of ferrocarbon particles is maintained at a high level.

The iron:carbon particles are characterized by a well-developed substructure (see FIG. 1), having a connected network of iron 12 forming a network of voids with carbon deposits 10 captured therein. The characteristic substructure of the magnetically targetable carrier particles formed during the process of joint deformation of the mechanical mixture of iron and carbon powders, also increases the magnetic susceptibility of iron inclusions in ferrocarbon particles 8 as compared with iron particles having other types of substructure. For example, the composite ferrocarbon particles produced by the herein suggested method have greater magnetic susceptibility than the particles disclosed in European Patent Office Publication No. 0 451 299 A1, although the ferromagnetic content in both types of magnetically targetable carrier particles is about the same. This high magnetic responsiveness of ferrocarbon particles 8 makes it possible, in some cases, to utilize magnetic fields lower than about 250 gauss to position the particles at the desired anatomical site.

Because of the large surface of carbon deposits 10 in magnetically targetable carrier particles 8, the adsorbed biologically active substance comprises up to about 20.0% by mass of particles 8; or, in different terms, up to about 200 mg of adsorbed biologically active substance per gram of particles 8. Therefore, in use, much less of the carrier is injected to achieve a given dose of the biologically active substance or, alternatively, a higher dosage of the biologically active substance per injection is obtained than is the case with some previously known carriers.

In one embodiment, the creation of the magnetically targetable carrier particle is performed in ethanol and the milling is conducted with a high energy mill that generates an increased temperature and pressure. The carbon is dried before use to reduce the moisture content. The adsorptive power is tested with mitomycin after manufacture. In one embodiment, the iron and carbon used will have properties in addition to those that allow the magnetically targetable carrier particle to be magnetically targetable and absorb and release a biologically active sample. For example, in one embodiment, it may be desirable that the iron and carbon have the characteristics listed in Table 1. Type KB carbon is one such carbon which meets these criteria.

TABLE 1
Starting Materials and Associated Tests.

| Starting Material | Tests performed | Specification |
|---|---|---|
| Activated | Acid Soluble Substances | Meets USP |
| Charcoal | Chloride | Meets USP |
| | Cyanogen Compounds | Meets USP |
| | Heavy Metals | Meets USP |
| | Reaction | Meets USP |
| | Residue on Ignition | Meets USP |
| | Sulfate | Meets USP |
| | Sulfide | Meets USP |
| | Uncarbonized Constituents | Meets USP |
| | Identity | 95-105% Carbon |
| Iron | Appearance | Uniform powder |
| | Color | Gray |
| | Apparent density | 1.5 to 3.0 g/cc |
| | True density | 7.5 to 7.8 g/cc |
| | Sieve on 200 mesh | 0.2% maximum residual |
| | Average particle diameter | 1-3 μm |
| | % Iron | 98% |
| | % Carbon | <1% |
| | % Oxygen | <0.7% |
| | % Nitrogen | <1% |
| | Identity | Passes Test |

The activated carbon can be dried in a vacuum oven at 80° C. for a minimum of 42 hours and stored desiccated once dried. Activated carbon may be dried in sub-lots.

Dehydrated Alcohol, USP can be used as a solvent. Dehydrated alcohol USP can be used in the milling, separation, and homogenization steps. The magnetically targetable carrier particle can be packaged in a glove box overlaid with nitrogen. The specifications for the nitrogen used met or exceeded the requirements of the National Formulary (NF) with the omission of an odor test.

The following describes one embodiment of a method for producing small quantities of the ferrocarbon composition of this embodiment, it being understood that other means and mechanisms besides milling could be conceived of for jointly deforming iron and carbon powders, which comprise the essential starting elements for production of the carrier. The procedure utilized exerts mechanical pressure on a mixture of carbon and iron particles to deform the iron particles and develop a substantial substructure, which captures the carbon. The formation of the ferrocarbon particles is accomplished without the addition of heat in the process (although the mixture heats up during the mechanical deformation step), and is conducted in the presence of a liquid, for example ethanol, to inhibit oxidation of the iron and to assure that the particles produced are clean. The liquid may also serve as a lubricant during the milling of the iron and carbon powder, and may reduce compacting of carbon during processing. As a result, the density of the carbon deposits in the composition is maintained so as to maximize adsorption capacity of the magnetically targetable carrier particles.

For example, to produce magnetically targetable carrier particles having an average of about 75:25 iron:carbon ratio by mass, one part of substantially pure iron particles are milled with about 0.1 to 1.0 parts by weight of substantially pure carbon granules. In one embodiment, the diameter of 95% (by population) of the resulting magnetically targetable carrier particles is less than or equal to 5.00 microns and the mean diameter is 0.6 microns to 3.5 microns. The iron particles and carbon granules are milled vigorously to achieve good distribution throughout the volume. Preferably the carbon granules are activated carbon. Each biologically active substance should be evaluated individually with the various types of carbon in order to determine the optimum reversible activated carbon binding. Factors such as pH, temperature, particulate size, salt solution viscosity and other potentially competing chemicals in solution can influence adsorption capacity, rate, and desorption parameters. Activated carbon types that are useful include, but are not limited to A, B, E, and K types and specifically KB and chemically modified versions thereof. These particular types of activated carbon were discovered to have an unexpectedly high ability to bind biologically active substances. In one embodiment, the carbon content will be 18% to 25% of the total mass.

The mixture is put into a standard laboratory planetary ball, or attrition, mill of the type used in powder metallurgy. For example, the mill can have 6 mm diameter balls. An appropriate amount of a liquid, for example ethanol, is added for lubrication. The mixture is milled for between 1 and 12 hours, or for the time necessary to produce the magnetically targetable carrier particles heretofore described. Depending on the mill used, the speed of the mill may be anywhere in the range from about 120 rpm to about 1000 rpm (typically about 270-330 rpm).

After joint deformation of the iron:carbon mixture, the magnetically targetable carrier particles are removed from the mill and separated from the grinding balls, for example, by a strainer. The particles may be resuspended in ethanol and homogenized to separate the particles from each other. The ethanol is removed, for example, by rotary evaporation, followed by vacuum drying. Any suitable drying technique may be employed. Magnetically targetable carrier particles should be handled so as to protect against oxidation of the iron, for example, in a nitrogen environment. In one embodiment, the loss from drying is ≦5.0% by weight.

Magnetically targetable carrier particles can be tested for residual solvent. Total residual solvents are less than or equal to 5000 ppm.

After drying, the magnetically targetable carrier particles should be collected according to appropriate size. For example, the magnetically targetable carrier particles may be passed through a 20 μm sieve and collected in an air cyclone to remove particles larger than 20 μm. The cyclone only collects particles of a certain size and density, providing a method for removing fines and loose carbon. The sieved particles may be packaged under nitrogen and stored at room temperature.

Magnetically targetable carrier particles may be subaliquoted into dosage units, for example, between 50 and 500 mg per dose, and may be further overlaid with nitrogen, for example. Dosage units may be sealed, for example, with butyl rubber stoppers and aluminum crimps. Dosage units may then be sterilized by appropriate sterilization techniques, for example, gamma irradiation.

When ready for use, or before packaging if the carrier is to be prepared with a preselected biologically active substance already adsorbed thereon, about 50 mg to 150 mg of the biologically active substance in solution is added to 1 gram of the carrier. When ready for application to a patient, the combination is placed into suspension (for example, in 5 to 100 ml) of a biologically compatible liquid such as water or saline utilizing normal procedures, e.g. a delivery vehicle.

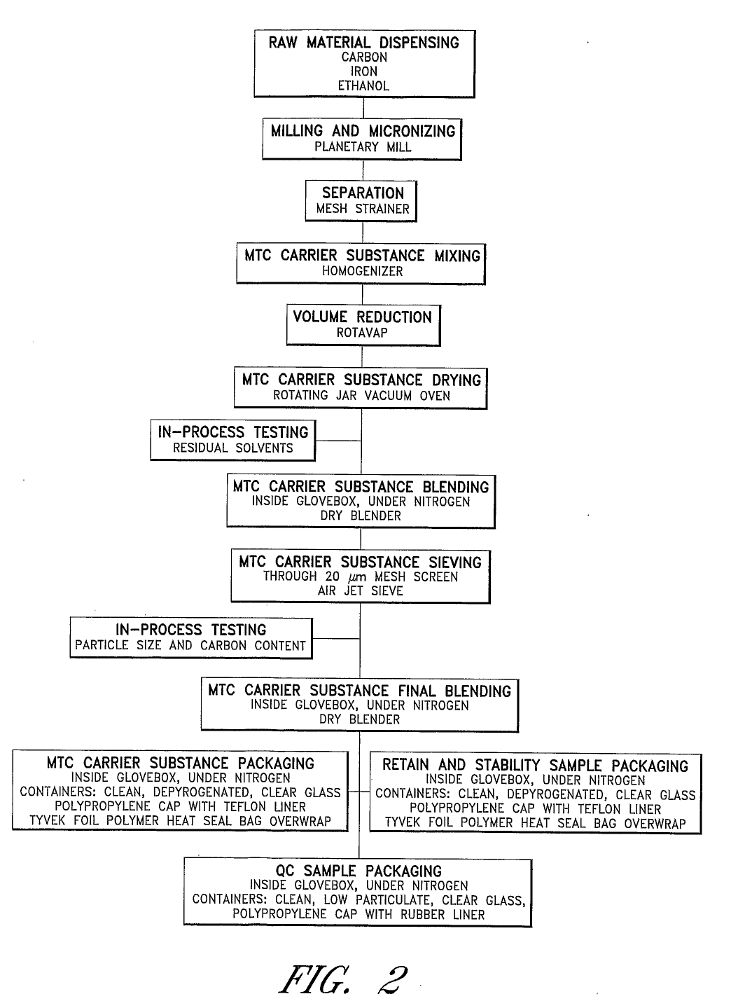

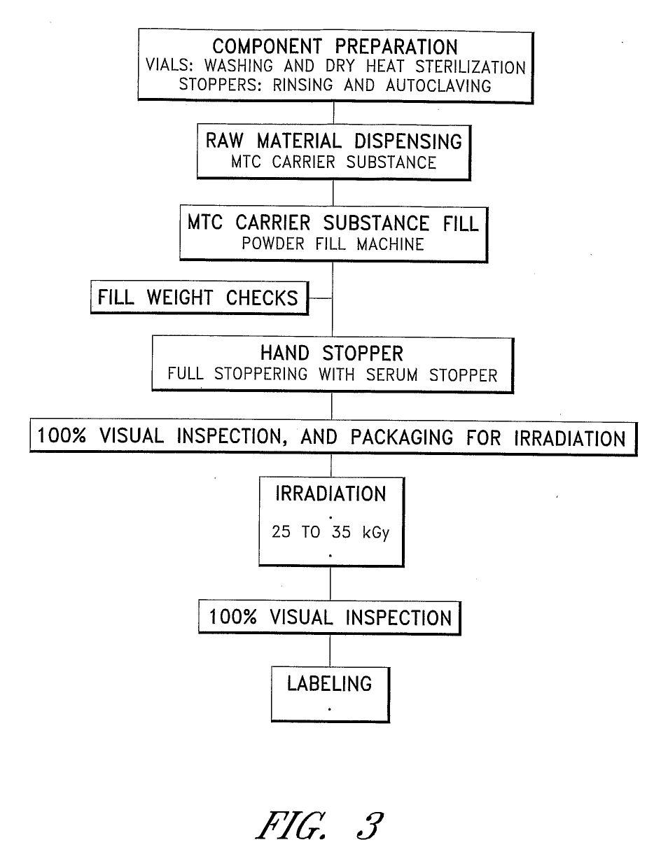

Flow charts describing two embodiments for methods for producing the magnetically targetable carrier particles are shown in FIGS. 2 and 3.

Preferred Magnetically Targetable Carrier Particles for Mitomycin C

In a preferred embodiment, the magnetically targetable carrier particle for adsorption with mitomycin C is a composite particle formed by mechanochemical milling of metallic iron and activated carbon (Volkonsky, et al., U.S. Pat. No. 5,651,989, (1997); Rudge et al., Biomaterials, 21, 1411-1420, (2000), both herein incorporated by reference in their entireties). In one embodiment, magnetically targetable carrier particles can be milled starting with 65% metallic iron powder and 35% activated carbon powder. The size of the resulting magnetically targetable carrier particles ranged from approximately 0.5 to 5 μm. The magnetically targetable carrier particles do not, per se, have a molecular structure or formula that describe their total composition.

The iron portion of the magnetically targetable carrier particle is a finely dispersed pure iron crystal that has a body centered cubic (bcc) crystalline structure. This structure is largely responsible for the magnetic response of the magnetically targetable carrier particle. Other forms of iron have much reduced magnetic saturation, as shown in Table 2. The table shows that it is desirable for the product to retain as large a percentage of crystalline iron as possible, in order to retain the highest possible magnetic saturation within the magnetic field imposed. This form of iron is also known as metallic iron.

TABLE 2
Magnetic Saturations of bcc Iron, Magnetically Targetable
Carrier Particle and Other Forms of Iron

| | Magnetic Saturation |
|---|---|
| Iron Form | (Am²/kg) |
| bcc iron | 218 |
| magnetically targetable carrier | 145.6 |
| particle substance, Lot | |
| Fe₃C | 128 |
| FeO•Fe₂O₃ | 98 |
| Fe₃O₄ | 92 |
| γFe₂O₃ | 74 |

By iron, what is meant is metallic iron. “Metallic iron” as used herein for the making of the magnetically targetable carrier particles is essentially chemically pure, with higher than about 85% atomic iron, and most preferably higher than about 90%. The iron used for making the magnetically targetable carrier particles used in this embodiment also typically contains less than about 20% iron oxides, more preferably less than about 10%, and most preferably less than about 5%, it being noted that the magnetically targetable carrier particles may contain impurities in addition to iron oxides. Metallic iron is a material with high magnetic saturation and density (218 emu/g and 7.8 g/cm³) which are much higher than magnetite (92 emu/g and 5.0 g/cm³). The density of metallic iron is 7.8 g/cm³, while magnetite is about 5.0 g/cm³. Thus, the magnetic saturation of metallic iron is about 4-fold higher than that of magnetite per unit volume. (CRC Handbook, 77th edition, CRC Press (1996-1997) and Craik, D., Magnetism Principles and Applications, Wiley and Sons (1995).

The activated carbon phase of the magnetically targetable carrier particle similarly has no defined chemical structure. Activated carbon is a macroporous substance composed primarily of aromatic carbon rings.

In one embodiment, the activated carbon raw material is formed by charring a natural fiber source, such as oak. The char is activated with hot phosphoric acid. The activated carbon is washed in water and size classified. In one embodiment, the activated carbon types described above are used.

The milling process employed in the manufacture of magnetically targetable carrier particles incorporates the activated carbon into the magnetically targetable carrier particle, without reducing its activation, that is, with the pores and pore structure largely intact.

Because ferric oxide (Fe₃O₄, magnetite) is less magnetically saturatable than metallic iron, protection of the iron portion of the magnetically targetable carrier particle from oxidation due to moisture or air is desirable. For this reason, all process steps, from milling through drying of the magnetically targetable carrier particle, are carried out in ethanol or under nitrogen.

In order to provide assurance magnetically targetable carrier particles greater than 20 μm are not packaged with the product, a sieving step is utilized for the manufacture of magnetically targetable carrier particle. The sieve employs a vacuum to pull magnetically targetable carrier particles through a 20 μm mesh, and uses a cyclone to collect sieved particles upstream of the vacuum.

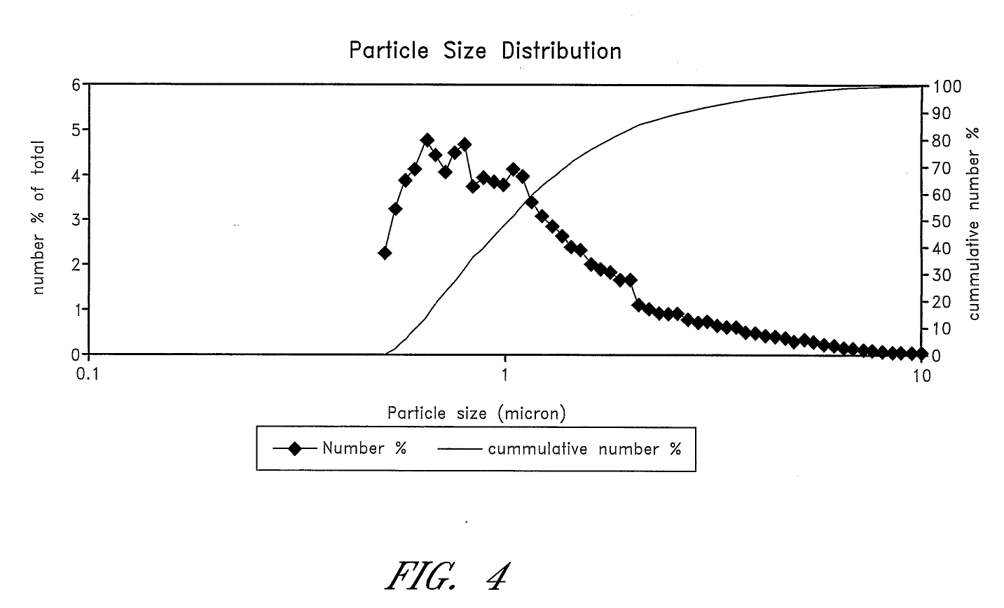

Magnetically targetable carrier particle size distribution was determined by laser light scattering from individually sorted particles with a PSS Accusizer 770 (Particle Sizing Systems, Santa Barbara, Calif.). Laser light scattering allows the sensor to count particles, based on the number of negative pulses detected in the light path, and on the height of the average of the scattered light intensities associated with that pulse. The resulting diameter is a spherical equivalent diameter for irregular or non-spherical particles. The sensor's range is 0.5 μm to 400 μm (nominal). In one embodiment, the magnetically targetable carrier particle size distribution of a magnetically targetable carrier particle sample typically ranges from 0.5 to 5 μm. A representative magnetically targetable carrier particle size distribution, by number of particles, is shown in FIG. 4.

In one embodiment, the diameter of 95% (by population) of the magnetically targetable carrier particles is less than or equal to 5.00 microns and that the mean diameter is 0.6 microns to 3.5 microns.

The magnetically targetable carrier particle surface area was determined by nitrogen gas adsorption. The surface area measured for one particular lot is 289 m²/g. The volume-weighted average pore size as measured by nitrogen adsorption is 4.2 nm.

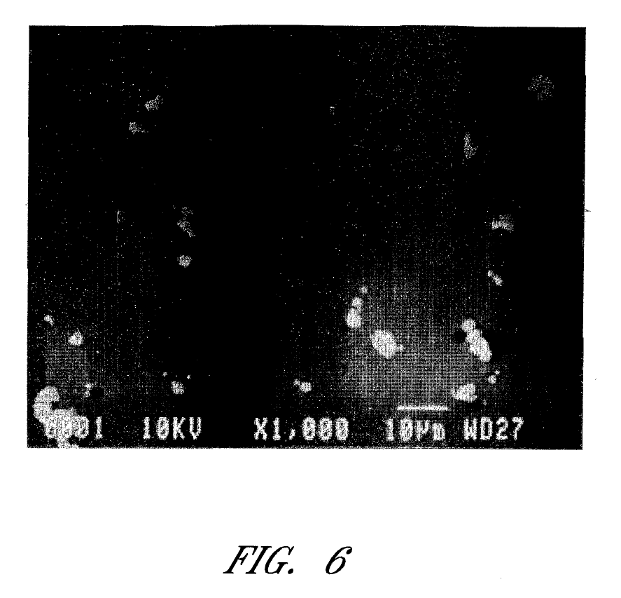

Electron microscopy was performed on a particular lot. Magnetically targetable carrier particles were resuspended in magnetically targetable carrier particle delivery vehicle, and sonicated before the pictures were taken. The micrographs show large numbers of 0.5 to 5 micron sized magnetically targetable carrier particles of an irregular shape. In one embodiment, all micrographs show a preponderance of single magnetically targetable carrier particles 1 to 5 μm in size, and all particles less than 10 μm. FIG. 6 shows magnetically targetable carrier particles at 1000× magnification in a backscatter image. The image reveals in dark, the outline of the carbon and in light, the iron.

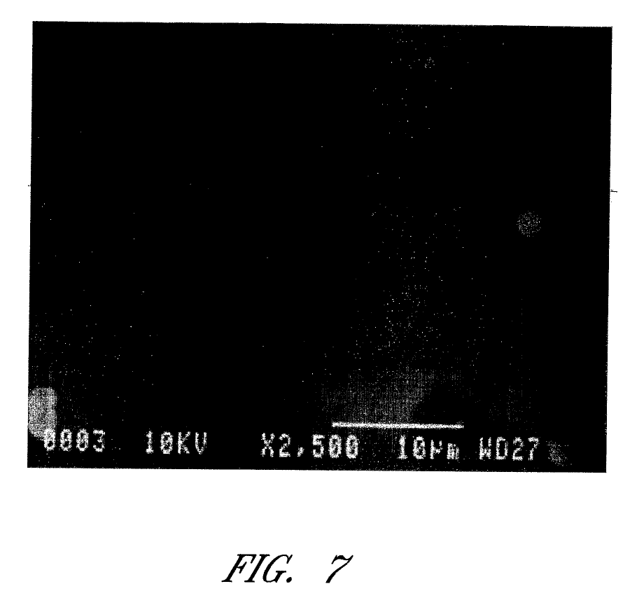

Further magnification, as shown in these micrographs (FIGS. 7, 8, and 9) demonstrate that the irregular morphology and mixed composition of the magnetically targetable carrier particles are maintained across particle sizes.

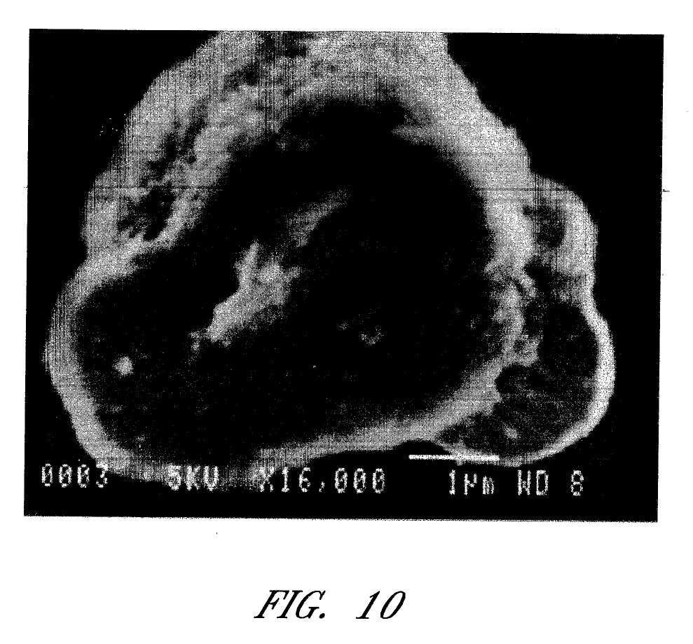

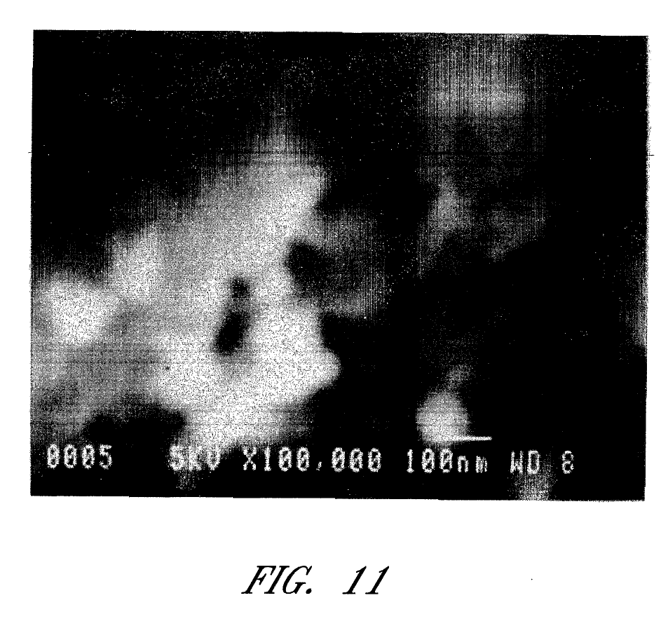

FIGS. 10 and 11 illustrate the image of a single magnetically targetable carrier particle to further elucidate the composition and structural information of an magnetically targetable carrier particle, and confirm that the particles are composed of distinct solid phases of activated carbon and metallic iron, and that the pore structure of the carbon is conserved after milling. The surface area of the carbon is important for the adsorptive properties of the magnetically targetable carrier particles.

In one embodiment, the magnetic susceptibility of the magnetically targetable carrier particle can be measured by suspending a sample of the material in a magnetic field, and measuring the extent of the orientation of the sample's magnetic vectors in the direction of the axis of the magnetic field. This can be done, for example, by introducing the sample into a vibrating magnetometer (LakeShore Cryotronics model 7404, Westerville, Ohio).

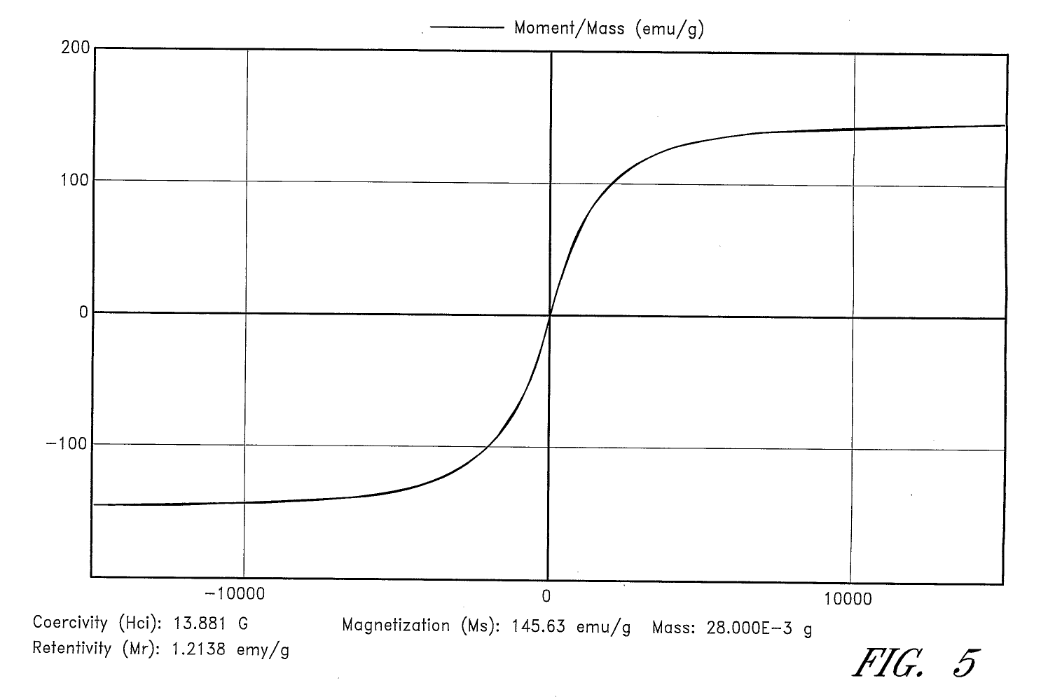

For this example, magnetic saturation occurred at approximately 145.6 A·m²/kg. When compared to other iron-containing materials, the magnetically targetable carrier particle's magnetic saturation is second only to bcc iron, as shown in Table 2. The magnetic saturation measured for the magnetically targetable carrier particle (FIG. 5) is consistent with what would be expected for a material containing approximately 75% bcc iron.

In one embodiment, the magnetically targetable carrier particle is packaged under nitrogen to protect the substance from moisture and oxygen. In another embodiment, magnetically targetable carrier particles are packaged under atmosphere containing less than 60% r.h. to reduce the redox potential of the particle.

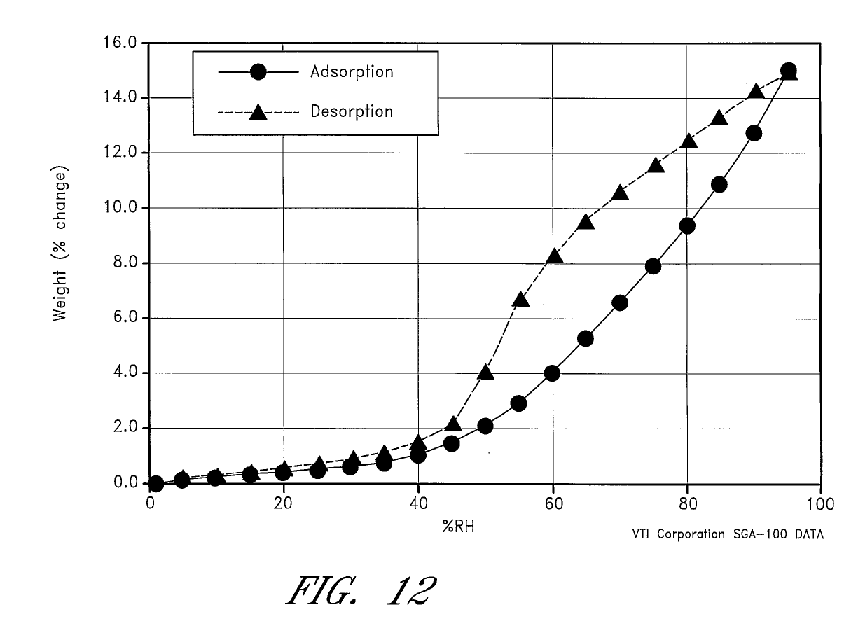

In one embodiment, the magnetically targetable carrier particle can be packaged under nitrogen to protect the substance from moisture and oxygen. To determine the effects of humidity on the magnetically targetable carrier particle, the water adsorption isotherm for magnetically targetable carrier particle can be determined by gravimetric adsorption. Magnetically targetable carrier particles do not adsorb moisture appreciably at relative humidity below 50%, as shown in FIG. 12. Activated carbon has a primarily hydrophobic surface that requires significant activation energy to adsorb the first layer of water. After adsorption of the monolayer, subsequent layers adsorb with lower activation energies, leading to the “concave upwards” isotherm. The low weight gain below 50% r.h. indicates that magnetically targetable carrier particles may be handled in atmospheres between 0% and 50% r.h. without significantly affecting their properties. The desorption cycle indicates hysteresis at higher relative humidities. Hysteresis is sometimes caused by molecular rearrangement after adsorption, indicative of multiple mechanism of adsorption. The weight gain appeared reversible.

The characterization data provided above demonstrate that in one embodiment, magnetically targetable carrier particles have porosity and adsorptive properties similar to those of activated carbon, as well as metallic iron properties, demonstrated by magnetically targetable carrier particle magnetization. Electron microscopy confirms the presence of composite iron/carbon particles largely between 0.5 and 5 microns. By these measures, the magnetically targetable carrier particle is capable of performing its intended purpose of carrying mitomycin to tumor sites under the influence of a magnetic field.

Packaging of Magnetically Targetable Carrier Particles

In one embodiment, the magnetically targetable carrier particles produced above are filled as 150 mg magnetically targetable carrier particle powder aliquot in 50 mL vials. The vials are stoppered under an atmospheric headspace environment. The product is rendered sterile by gamma irradiation. In one embodiment, lots of magnetically targetable carrier particle are filled into 50 ml cleaned depyrogenated Type I borosilicate vials in a Class 100 laminar flow hood in a class 1000 environment. The vials are stoppered with clean, serum stoppers. The vials are then sealed with aluminum crimps, visually inspected for defects and packaged for irradiation. Vials are stored and shipped at room temperature. Irradiation is performed. The vials absorb a sterilizing dose, for example, 25 kGy from a cobalt source.

In one embodiment, magnetically targetable carrier particle vials contain 150±7.5 mg of the magnetically targetable carrier particle. In order to ensure that 150 mg of magnetically targetable carrier particle is available for adequate adsorption at the intended clinical load of mitomycin C, a 7.5 mg “overfill” of magnetically targetable carrier particles in the vial has been established. In one embodiment, the fill weight is 157±7.5 mg overfill.

In one embodiment, the magnetically targetable carrier particles are also lyophilized. They may be lyophilized with or without excipients and with or without MMC and/or other biologically active substances. Following this they may be loaded into a vial, any headspace which remains can be backfilled with nitrogen, air oxygen, or any appropriate mixture.

Analysis of Magnetically Targetable Carrier Particles

In one embodiment, the magnetically targetable carrier particles are analyzed after they are created. In this embodiment, the iron content should be about 64% to 86% of the total mass, the carbon content should be about 18% to 25% of the total mass, the amount of iron as iron oxide should be about less than 5% of the atomic iron. Additionally, the specification for loss on drying is about ≦5.0% by weight. To measure heavy metals, samples can be digested and then ionized in an inductively-coupled plasma interfaced with a Perkin Elmer Elan 5000 mass spectrometer. The approximate limit of detection for the mass spectrometer is 0.1 ppb. The specification for heavy metals is listed below in Table 3.

TABLE 3
Heavy Metals Specification

| Element | Acceptance Criteria |
|---|---|
| Sodium | ≦10,000 ppm |
| Phosphorous | ≦10,000 ppm |
| Silicon, Chromium, | Total of 3 elements ≦3,000 ppm, |
| and Nickel | with no individual result >1,000 ppm |
| Mn, Mo, Al, Ti, Cu, Hg, | Total of 13 elements ≦1,000 ppm, |
| Bi, As, Pb, Sb, Sn, | with no individual result >100 ppm |
| Cd and Ag | |

In one embodiment, the magnetically targetable carrier particle should be depyrogenated and contain ≦0.2 EU/mg. Table 4 contains release results for the two representative magnetically targetable carrier particle lots.

TABLE 4
Release Testing Results for two Magnetically
Targetable Carrier Particle Lots

| Test | lot 1 | lot 2 |
|---|---|---|
| Color | conforms | conforms |
| Appearance | conforms | conforms |
| Magnetically targetable | 2.73 | 2.21 |
| carrier particle | | |
| size (95% < diameter) (μm) | | |
| Magnetically targetable | 1.12 | 1.02 |
| carrier particle | | |
| size (mean diameter) (μm) | | |
| Iron % | 73% | 74% |
| Carbon % | 20% | 21% |
| Iron Oxide atomic % | 1% | 1% |
| Total Residual Solvents % | 0.2% | 0.2% |
| Loss on Drying (%) | <1.5 | <1.5 |
| Heavy Metals | conforms | conforms |
| Identity | conforms | conforms |
| Endotoxin | 0.0 EU/mg | 0.0 EU/mg |

In one embodiment, the residual solvents, such as ethanol content can be measured as an end point to the magnetically targetable carrier particle drying step, the carbon content and magnetically targetable carrier particle size can be tested during the magnetically targetable carrier particle sieving step.

In one embodiment, an assay to measure the total binding capacity of magnetically targetable carrier particle to bind mitomycin can be used.

Making Magnetically Targetable Carrier-MMC Particle

In one embodiment, the magnetically targeted carriers with mitomycin C adsorbed onto the carriers (magnetically targetable carrier-MMC particle) are produced as follows. Mitomycin, USP was supplied as a sterile dry mixture of 20 mg mitomycin and 40 mg mannitol in 50 mL vials. To formulate magnetically targetable carrier-MMC particle, mitomycin was reconstituted with 26.7 mL Sterile Water for Injection to bring the concentration of mitomycin to 0.75 mg/mL and mannitol to 1.5 mg/mL. This is in contrast to the conventional methods, where 40 mL Sterile Water for Injection would be added to produce 0.5 mg/mL MMC and 1 mg/mL mannitol. In one embodiment, the ratio of combined weight of iron and carbon to MMC is about 1:100 to about 10:100. In a preferred embodiment, the ratio is 5:100.

As appreciated by one of skill in the art, the ratios between carbon iron, and between total carbon and iron to mitomycin C are different. When the ratio concerns a comparison between carbon and iron, the relative amounts are out of 100%. On the other hand, when MMC is being compared to iron and carbon, the relative amounts are strictly in relation to each other; thus, for example, 10 parts by weight MMC to 100 parts by weight of the iron and carbon magnetically targetable carrier particles.

Delivery Vehicle

In one embodiment, the magnetically targetable carrier particle can be associated with a delivery vehicle. By delivery vehicle, what is meant is a substance to which the magnetically targetable carrier particles may be added, in order to deliver the magnetically targetable carrier particles to a patient. In one embodiment, the delivery vehicle is provided as a medium of sufficient viscosity to retard the settling of the drug-laden carrier while sufficiently fluid to maintain good infusion properties. In one embodiment, the delivery vehicle can be provided as a sterilized solution in a Type I borosilicate clear glass 50 and 30 mL vial. Table 5 lists the contents per vial for the magnetically targetable carrier particle delivery vehicle.

TABLE 5
Contents per Vial for Magnetically Targetable
Carrier Particle Delivery Vehicle

| Lot | Vial | Label | |
|---|---|---|---|
| Number | Size (mL) | Claim (ml) | Contents per gram |
| A | 50 | 40 | 100 mg Mannitol USP |
| | | | NLT 3 mg Carboxymethylcellulose, |
| | | | Sodium, USP |
| | | | Water for Injection USP to 1 g |
| B | 30 | 21 | 100 mg Mannitol USP |
| | | | 5 mg Carboxymethylcellulose, |
| | | | Sodium, USP |
| | | | Water for Injection USP 895 mg |

The following is one embodiment by which the magnetically targetable carrier particle delivery vehicle can be made. On a weight basis, a 10% Mannitol, USP, 0.5% Sodium Carboxymethylcellulose, USP, solution was prepared in Water for Injection, USP. In one embodiment, the Mannitol is a solubilizing agent (i.e., excipient). However, in one embodiment, any compound that can serve to promote the solubility of the magnetically targetable carrier particle could also be used. The viscosity of the solution was approximately 14±2 cP when measured at 40° C. and 60 rpm in a Brookfield viscometer. The magnetically targetable carrier particle delivery vehicle was filtered through a 5 μm/1 μm/0.45 μm filter train into an air classified room. Magnetically targetable carrier particle delivery vehicle was filled in a class 100 environment into 30 mL cleaned depyrogenated vials. Vials are stoppered and sealed, then terminally sterilized in an autoclave. The autoclave cycle is 30 minutes at a minimum of 121° C.

Alternatively, the solution can be prepared initially with only 0.3% Sodium Carboxymethylcellulose USP. Then the viscosity of the solution is adjusted with a 3% Sodium Carboxymethylcellulose, USP, 10% Mannitol USP solution that is also prepared with Water for Injection, USP. The autoclave can be used for 40 minutes, >121° C. A water cascade can be used to cool vials at the end of the autoclave cycle. Many other methods of manufacture would be apparent to one skilled in the act in light of this application.

In one embodiment, the delivery vehicle's osmolality is 610-650 m/Osm/kg.

In one embodiment, the delivery vehicle's viscosity is tested in a calibrated Brookfield type cone and plate viscometer with a #TL5 spindle at 60 rpm and 40° C. One preferred viscosity is 6-12 cP.

In one embodiment, a bacterial endotoxin testing can be performed. In one embodiment, the endotoxin limit is ≦2.8 EU/mL.

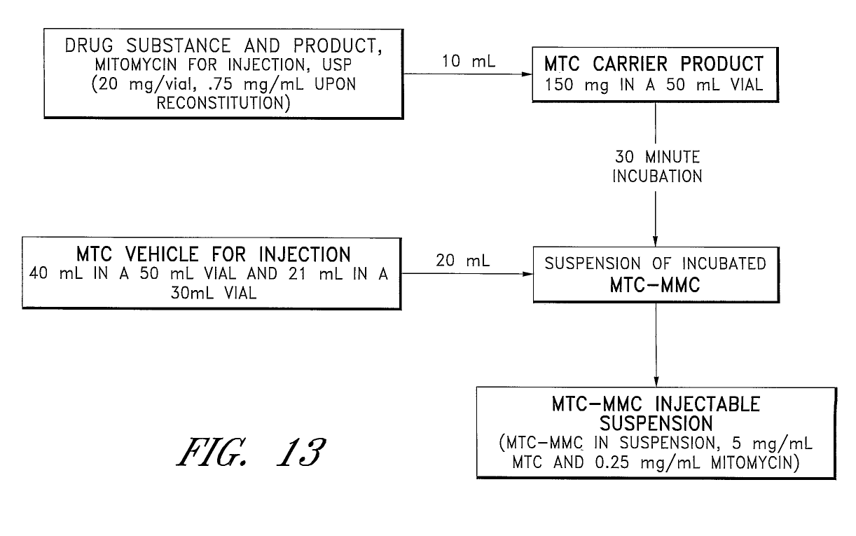

In one embodiment, magnetically targetable carrier-MMC particle is a combination of mitomycin with mannitol, magnetically targetable carrier particle and magnetically targetable carrier particle delivery vehicle. Mitomycin (20 mg Mitomycin for Injection and 40 mg mannitol in 50 mL vials) can be reconstituted at 0.75 mg/mL by adding 26.7 mL Sterile Water for Injection. 10 mL of the reconstituted mitomycin can be added to the magnetically targetable carrier particle packaged as 150 mg in a sterile vial, and incubated for thirty minutes at room temperature. Mitomycin adsorbed to magnetically targetable carrier particles is called magnetically targetable carrier-MMC particle. After the adsorption step, 20 mL of magnetically targetable carrier particle delivery vehicle can be added. Mitomycin can be adsorbed to the magnetically targetable carrier particles at about 5% weight to weight, so the resulting magnetically targetable carrier-MMC particle concentration in suspension is about 0.25 mg/mL mitomycin with an magnetically targetable carrier particle concentration of about 5 mg/mL. The required dose is withdrawn from the vial and syringe mixed prior to administration. One embodiment for the method of producing the magnetically targetable carrier-MMC particle suspension is shown in FIG. 13.

Once the combination of magnetically targetable carrier-MMC particle and a delivery vehicle (i.e., excipient) is created, the formulation can be administered to a patient. In order to localize the magnetically targetable carrier-MMC particle to the desired location a non-alternating magnetic field is established exterior to the body and adjacent to the targeted site, and having sufficient field strength to guide a substantial quantity of the injected magnetically targetable carrier particles to, and retain the substantial quantity of the particles at the site. This field may be established just prior to, during or after injection. Preferably, the magnetic field is of sufficient strength to draw the magnetically targetable carrier particles into the soft tissue at the site adjacent to the network of vessels, thus avoiding substantial embolization of any of the larger vessels by the particles, should embolization be undesirable for the particular treatment/diagnosis. Examples of such magnets for use in the instant methods are those producing at least about 100 gauss of non-alternating magnetic flux at the region of interest (target site), the exact magnetic field strength being dependent upon the application, for instance the blood flow rate, the thickness of the endothelium, and the depth and diffuseness of the tumor tissue. For example, a NdFeB magnet producing a flux of about 5 kG at its N pole surface, having a dimension of about 5 cm diameter, 6 cm length, can be used to direct magnetically targetable carrier particles described herein in both healthy and diseased liver tissue. (Part No. MSD12691-NC, Magnet Sales, Culver City, Calif.). Other compositions of NdFeB, and other rare earth, ceramic, or electromagnets or superconducting magnets may also be suitable.

There are many alternative mechanisms for guiding the magnetically targetable carrier particles to the desired region in the host. Which approach is desirable for a given situation will depend upon the goal to be achieved, given the present disclosure, one of skill in the art will be able to readily determine which approach should be used. In one embodiment, the magnetically targetable carrier particles are directed and controlled by the invention of Mitchiner et al., U.S. Pat. No. 6,488,615, issued Dec. 3, 2002, Mitchiner et al., U.S. Pat. No. 6,663,555, issued Dec. 16, 2003, and U.S. patent application Ser. No. 10/734,651 filed, Dec. 12, 2003 to Thomas Kent, herein incorporated in their entireties by reference. This reference provides both the device for administering a magnetic field to a patient in order to capture these particles, and the method for doing so. Briefly, the device is a magnet keeper-shield assembly adapted to hold and store a permanent magnet used to generate a high gradient magnetic field. Such a field may penetrate into deep targeted tumor sites in order to attract magnetically targetable carrier particles. The magnet keeper-shield assembly includes a magnetically permeable keeper-shield with a bore dimensioned to hold the magnet. An actuator is used to push the magnet partially out of the keeper-shield. The actuator is assisted by several springs extending through the base of the keeper-shield.

The magnetically targetable carrier particles can be administered to a patient through catheterization. The practice of catheterization is well known in the art and the appropriate placement of a catheter in order to treat an organ is also well known. Catheter placement, though any means, either through intra-vascular or cannulation techniques, for example, is something that would be known to one of ordinary skill in the art

A toxicity study can be used to evaluate the safety profile of magnetically targetable carrier-MMC particle after it is made, for example, as shown in Example 7, which was in a swine model. In Example 7, targeting was demonstrated largely by histopathological examination of magnetically targetable carrier particle presence that was restricted to the site of delivery in the left lower lung lobe. Regions of healthy lung tissue had to be targeted in this study due to the lack of a relevant large animal lung tumor model. Magnetically targetable carrier particles would be expected to more readily extravasate from tumorous tissue that is often hypervascular. Consideration needs to be made for the potential differences in magnetically targetable carrier-MMC particle effects on normal and neoplastic cells. The lack of tumors may have contributed to the fact that there were measurable levels of circulating MMC following magnetic targeted delivery of magnetically targetable carrier-MMC particle delivery. Circulating blood levels of MMC were still nearly 50% lower in magnetically targetable carrier-MMC particle treated animals compared to MMC control animals, suggesting that the drug was in contact with the targeted site for a longer period of time in magnetically targetable carrier-MMC particle treatment groups. The amount of MMC measured at each time point and over the entire 3 hour sampling period was less for magnetically targetable carrier-MMC particle than MMC alone. MMC has been shown to accumulate in malignant tissue more than in normal tissues even after intravenous administration, and it is mainly inactivated in the liver, spleen, kidneys, brain, and heart (Fujita, Jap. J. Clin. Oncol. 12:151-162, 1971).

In one embodiment, the magnetically targetable carrier-MMC particle is delivered to a lung tissue through the pulmonary artery. Such a procedure is shown in Example 7.

The method of intra-arterial infusion of chemotherapeutic agents to treat lung tumors is a possibility for use in humans. In Hellekant et al., (Acta Radiol Diagn (Stockh), 20:478-496, 1979) bronchial angiography was performed in 9 patients with squamous cell carcinoma of the lung. Subsequently, 10 mg of MMC diluted with saline was administered intra-arterially. After 28 to 48 days, complete remission of the tumor occurred in 2 patients and partial remission occurred in 2 patients. In another study, Hellekant performed bronchial angiography and intra-arterial infusion of MMC in 39 patients with bronchogenic carcinoma (Hellekant 1979). No neurologic complications occurred, and the side effects were insignificant. The method appears to be of therapeutic value in the treatment of bronchiogenic carcinomas. In a study conducted by Elkolm et al., 51 patients with squamous cell carcinoma or adenocarcinoma were treated with selective intra-arterial administration of 4 infusions of MMC with 2- to 3-week intervals (Ekholm et al., Ann Radio (Paris), 23:346-348, 1980). On each occasion patients received 10 mg of MMC diluted in 100 mL of saline, which was infused into the bronchial artery at a rate of 5 to 7 mL per minute. Among these patients that received a full series of 4 treatments, a 50% decrease of the tumor area was demonstrated in 5 patients and a 20% to 50% decrease was demonstrated in 3 patients.

In one embodiment, the magnetically targetable carrier-MMC particles result in a minimal to 20 percent decrease in tumor area while reducing the exposure of healthy tissue to MMC. In another embodiment, the magnetically targetable carrier-MMC particles result in a 20-50% decrease in tumor area, while reducing the exposure of healthy tissue to MMC. In another embodiment, the magnetically targetable carrier-MMC particles result in a 51%, 52-59%, 58-70%, 70-80%, 80-90%, 90-99%, near complete, or complete reduction in tumor size. In one embodiment, each dose is between 15 mg to 150 mg of magnetically targetable carrier-MMC particles. In another embodiment, the dosage is up to 20 mg/m² of MMC, where a typical American male is 2 m². In one embodiment, the dosing frequency is 3 to 8 weeks. In one embodiment, a full hematological screening may be performed to determine if dosing should continue or be reduced.

The safety and targeting efficiency seen in the present examples, example 7, for example, suggest that the magnetically targetable carrier-MMC particles will function for their intended purpose and can be administered as described herein. There was no mortality or clinical signs. Changes in clinical pathology parameters were generally minor and transient. Circulating levels of MMC were reduced in the animals receiving magnetically targetable carrier-MMC particle. Treatment-related effects seen upon histopathological examination were limited to targeted lung where magnetically targetable carrier particles were observed in alveolar capillaries. One animal from the high dose group of magnetically targetable carrier-MMC particle had multiple pulmonary granulomas containing magnetically targetable carrier particles. Magnetically targetable carrier particles were not observed outside of the targeted lung regions. Specifically, no magnetically targetable carrier particles were noted within the reticulo-endothelial system. Pulmonary artery delivery of magnetically targetable carrier-MMC particle resulted in site-specific localization of magnetically targetable carrier particles in targeted lung lobes. Biologically significant gross and microscopic lesions were limited to the targeted areas of lung in a single animal administered magnetically targetable carrier-MMC particle containing 80 mg magnetically targetable carrier particle and 4 mg MMC. Magnetic targeting leads to high local levels of MMC over a more prolonged period, which should increase anti-tumor efficacy. In addition, in one embodiment, this method of administration reduces systemic exposure to MMC, thereby decreasing those adverse effects that would be observed after systemic administration. Therapy with magnetically targetable carrier-MMC particle will be especially useful in patients with inoperable NSCLC as a consequence.

Experimental evidence shows increased therapeutic efficacy on a tumor growth with the use of the magnetically controlled carrier composition of this embodiment with an anti-tumor preparation in comparison with previously known magnetically controlled dispersions.

The following examples are for demonstration purposes only and are not meant to limit the embodiments of the inventions unless so explicitly stated.

### EXAMPLES

### Example 1

The following process provides one example for the manufacture of magnetically targetable carrier particle. The manufacturing process to produce the magnetically targetable carrier particle consists of 10 steps:

Raw Material Dispensing: Iron and carbon powders, Dehydrated Alcohol USP and 4 mm milling balls are combined in a grinding canister.

Milling and Micronizing: Milling occurred in a planetary mill in grinding canisters. The grinding canisters and balls are made of chromium-hardened stainless steel. The material was milled at 270 to 330 rpm for 200 to 240 minutes, and then, milled at 120 rpm for 160 to 200 minutes.

Separation: After the milling process, the contents of the milling canisters were emptied into a strainer held over a collection dish. The milling canister contents were passed over the strainer to separate the raw magnetically targetable carrier particle from the balls. Dehydrated Alcohol USP was used to rinse the canisters and milling balls. The strainer and collection dish used for this step is stainless steel. After collection, the magnetically targetable carrier particle slurry was transferred into a glass container, and is diluted with Dehydrated Alcohol, USP.

Mixing: The magnetically targetable carrier particle slurry was homogenized at a speed of 7000±500 rpm for 5 minutes using a homogenizer. This step dispersed the magnetically targetable carrier particles into Dehydrated Alcohol USP as individual particles. The remaining steps in the process served to disperse the magnetically targetable carrier particles into a dry powder.

Solvent Evaporation: The magnetically targetable carrier particle slurry was transferred to a glass vacuum flask. Under a vacuum of 15-20 inches Hg, the dehydrated alcohol/magnetically targetable carrier particle slurry from the mixing step is rotary-evaporated at 60 to 70° C. to reduce the volume. Rotary evaporation continued until solid material pulled away from the sides of the evaporation flask.

Drying: When rotary evaporation was complete, the material was transferred to a rotating evaporation drum. Final drying was performed in a nitrogen-purged vacuum oven at 75 to 80° C. under at least 24 inches Hg vacuum.

Blending: The dried magnetically targetable carrier particle was transferred to a nitrogen-purged glovebox. The magnetically targetable carrier particle was blended for 2 minutes in a Waring blender at approximately 11,500 rpm.

Sieving: The blended magnetically targetable carrier particle was passed through a 20 μm mesh sieve screen under 6 to 12 inches of water vacuum. A cyclone downstream of the sieve separates ultra-fine particulate matter from the product fraction. The process gave an absolute cut off of 20 μm for magnetically targetable carrier particles on the high end of the size distribution and reduced particles on the low end of the size distribution in the cyclone.

Final Blending: The product fraction of the sieved magnetically targetable carrier particle was transferred to a nitrogen-purged glovebox and blended for twenty minutes in a Waring blender at approximately 7,000 rpm. The final blend process ensured a uniform powder after the sieving process.

Packaging: The material was then transferred to a nitrogen purged glove box for dispensing into containers. Samples were removed for testing, (including stability and retains as appropriate, for example), and the remaining magnetically targetable carrier particle is dispensed into storage containers. All handling of the magnetically targetable carrier particle during packaging is performed inside a nitrogen-purged glovebox.

### Example 2

This example demonstrates one embodiment of how one may prepare the magnetically targetable carrier-MMC particle compositions. Magnetically targetable carrier-MMC particle was prepared by incubating 10 mL of reconstituted MMC (0.75 mg/mL; Bedford Laboratories, Bedford, Ohio) with sterile magnetically targetable carriers packaged in vials containing 150 mg of magnetically targetable carrier-MMC particles at room temperature for 30 min. The suspension was diluted with 20 mL of a viscous mannitol and carboxymethylcellulose buffer so that MMC was adsorbed to magnetically targetable carrier particles at a 5% (wt:wt) concentration.

### Example 3

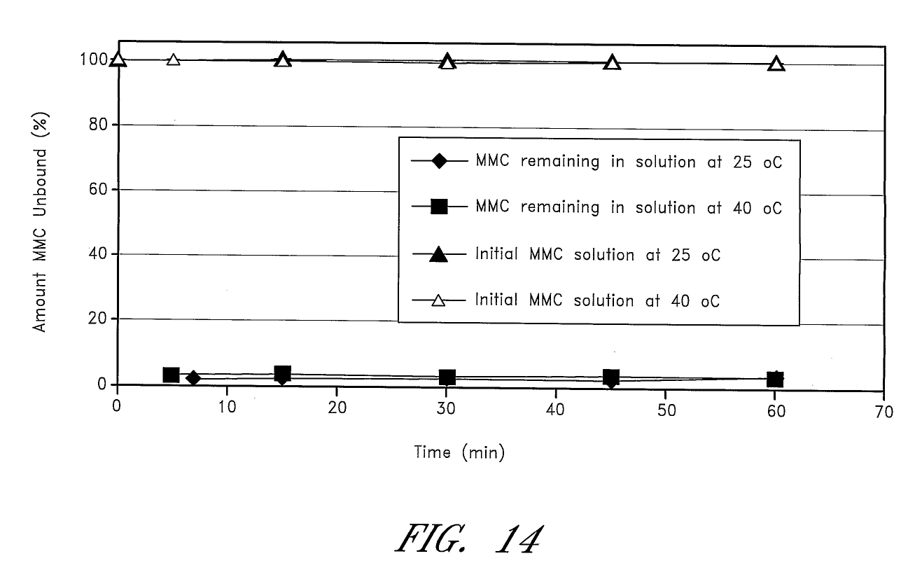

This example demonstrates how one can determine how much MMC has bound to the magnetically targetable carrier particle. Mitomycin binding kinetics was measured at room temperature, and 40° C. 25 mg magnetically targetable carrier particle was mixed with 1.66 mL of 0.755 mg/mL mitomycin solution to achieve a MMC load of 5.6%. The mixture was incubated at room temperature and at 40° C. for various time periods up to 60 minutes. The concentration of mitomycin remaining in the binding supernatant was obtained by filtering the sample at a predefined time point and then analyzing by HPLC to determine percentage bound. The percentage of mitomycin remaining in the binding supernatant versus the time of incubation is shown in FIG. 14.

The results indicate that in 5 minutes, 2% of the initial mitomycin remained in the supernatant at room temperature and 3% remained at 40° C. The adsorption reached equilibrium in less than 5 minutes at both room temperature and 40° C. indicating adsorption occurs rapidly. The mitomycin solution is stable at both temperatures and no significant difference is observed during the incubation period. This indicates that the adsorption appears to be rapid and temperature-independent.

### Example 4

The following example demonstrates how one may determine the dose accuracy of magnetically targetable carrier-MMC particle-delivery vehicle. 10 mL of MMC solution at a concentration of 0.83 mg/mL was added into a vial of magnetically targetable carrier particle (150 mg) and thoroughly mixed. The mixture was incubated at room temperature for 30 minutes with periodic agitation. 20 mL of vehicle was then added to the vial to suspend the magnetically targetable carrier-MMC particles to achieve a volume of 30 mL and a final magnetically targetable carrier particle concentration of 5 mg/mL and MMC concentration of ˜0.25 mg/mL. Six vials were produced and at predefined time points of 1 hour and 6 hours, the suspensions were syringed flush 5 times immediately before infusion using a syringe pump. 10 mL of each suspension was infused through a saline-filled microcatheter at 120 mL/hr using a syringe pump. The empty syringe was then flushed with 5 mL of saline, which was pump-infused through the catheter at 120 mL/hr. Samples were collected and analyzed for particle concentration by absorbance at 600 nm. The dose accuracy was calculated by comparing the concentrations of magnetically targetable carrier-MMC particle in the infused suspension to those in the suspension without infusing through a catheter.

The recovery of magnetically targetable carrier-MMC particles was 96-106% and there was no significant difference in the recovery of the magnetically targetable carrier-MMC particle over time.

### Example 5

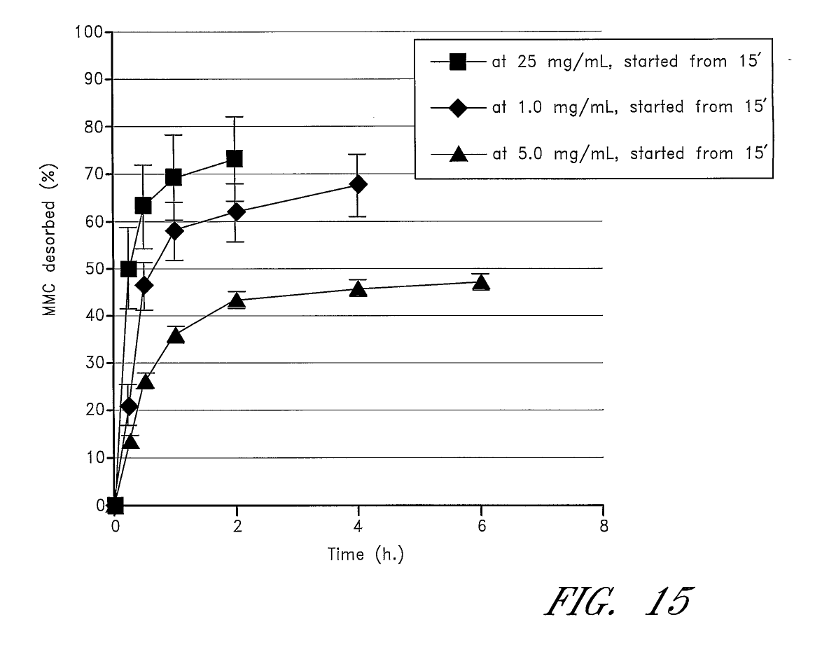

The following example demonstrates that MMC becomes bio-available after contact of magnetically targetable carrier-MMC particle with a biologically derived medium. The desorption of mitomycin from magnetically targetable carrier-MMC particle in human plasma was measured. Reconstituted mitomycin was incubated with 25 and 10 mg magnetically targetable carrier particles at room temperature for 30 minutes to produce magnetically targetable carrier-MMC particle with approximately 5%, by weight, adsorbed MMC. After centrifugation and removal of the adsorption supernatant, the magnetically targetable carrier particles were resuspended in human plasma at 37° C. At predefined time points, plasma supernatant was removed and replaced with the same volume of fresh plasma. The desorption study was performed at different ratios of magnetically targetable carrier particle to the volume of human plasma for up to 6 hours. Samples were extracted using acetonitrile to precipitate out proteins and then diluted with 1 mg/mL Mannitol solution and analyzed by HPLC. The amount of MMC desorbed into human plasma was calculated by comparison to a standard curve. The desorption profile revealed that that the desorption of MMC was initially rapid and then occurred more slowly after 2 hours. The desorption of MMC appeared higher with a lower ratio of magnetically targetable carrier particle to the volume of human plasma as indicated in FIG. 15. Up to ˜75% of the adsorbed MMC was desorbed from magnetically targetable carrier particles in human plasma.

The desorption profile shows that mitomycin can be released from magnetically targetable carrier particles in a biological fluid such as plasma. Mitomycin desorbed as a single pure peak with a retention time consistent with the mitomycin standard. Release is not immediate, but depends on the relative ratio of magnetically targetable carrier-MMC particle to plasma.

### Example 6

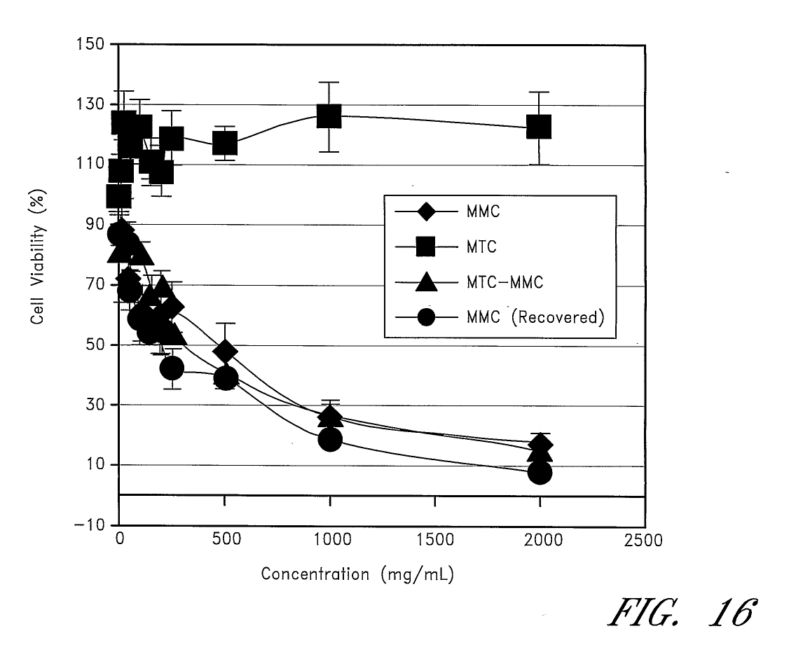

The following example demonstrates that magnetically targetable carrier-MMC particle retains the activity of MMC alone, as measured by cytotoxicity. In particular, this method can demonstrate the superior benefits of using the magnetically targetable carrier particle based approach of administering MMC to a patient. The cytotoxicity profile of magnetically targetable carrier-MMC particle in cell culture was analyzed using Human Non Small Cell Lung Cancer Cell line H460. MMC that was adsorbed onto magnetically targetable carrier particles was compared to MMC and magnetically targetable carrier particle alone as well as a pre-desorbed sample of MMC from magnetically targetable carrier particles. The cells were seeded into 96-well microtiter plates at 2000-4000 cells per well. Twenty-four hours after plating, known quantities of mitomycin, magnetically targetable carrier-MMC particle suspension, mitomycin desorbed from magnetically targetable carrier-MMC particle and magnetically targetable carrier particles alone were added. All treated cells were incubated for 24 hours, then a cell viability assay was performed. The MTT assay revealed that MMC, MMC released from magnetically targetable carrier particles and magnetically targetable carrier-MMC particle had a similar drug concentration resulting in cell death, while magnetically targetable carrier particle alone did not show any inhibition of cell growth (See Shetty et al., Journal of Postgraduate Medicine, 42:72-75, 1996, for a description of the MTT assay) (FIG. 16). All results are plotted on a MMC basis, except magnetically targetable carrier particles, which are plotted as if they were loaded with 5% MMC.

These results indicate that MMC remains functional as a cytotoxic agent and the potency is not altered through the adsorption and desorption processes.

### Example 7

The following example demonstrates how to administer and evaluate a toxicity profile of magnetically targetable carrier-MMC particle in the swine model. The example also provides a proof of principle for delivery of MMC and localization of MMC to a target area, e.g. the lungs, especially for human patients.

Safety of pulmonary artery delivery of magnetically targetable carrier-MMC particle was evaluated in a 28 day toxicity study conducted in 18 swine. MMC was adsorbed to magnetically targetable carrier particle microparticles containing iron that provided magnetic susceptibility. Pulmonary arteries were catheterized under fluoroscopic guidance for delivery of magnetically targetable carrier-MMC particle or magnetically targetable carrier particle or MMC alone to a specified lung area. An external magnet was used during administration to retain the test material in the targeted area. The animals were evaluated over 4 weeks for signs of toxicity.

Since there is no large animal lung tumor model large enough to accommodate pulmonary artery catheterization, effects were studied in normal swine because they show similarity to human vascular anatomy (McLaughlin, Am Rev Respir Dis, 128 (2 Pt 2):S57-8, 1983).

Female Yorkshire swine were selected for the study. Young adult animals were obtained from Pineview Farms, Valley City, Ohio and acclimated to laboratory conditions for a minimum of 7 days prior to study initiation. A total of 18 animals weighing 35±5 kg at the time of treatment were randomly assigned to 6 treatment groups of 3 animals each as described in Table 6. The test articles administered consisted of three different doses of magnetically targetable carrier-MMC particle and magnetically targetable carrier particle, MMC, and vehicle controls.

TABLE 6
TREATMENT GROUP ASSIGNMENTS AND DOSE LEVELS

| | | | Dose (mg/kg)¹ | | Total Dose (mg) | | |
|---|---|---|---|---|---|---|---|
| | | | MMC magnetically | | MMC magnetically | | |
| Group | Animals/ | | targetable carrier | | targetable carrier | | Dose |
| No. | Group | Treatment Group | particle | | particle | | Vol (mL) |
| 1 | 3 | magnetically targetable | 0.01 | 0.15 | 0.25 | 5 | 1² |
| | | carrier particle-MMC Low | | | | | |
| 2 | 3 | magnetically targetable | 0.03 | 0.62 | 1 | 20 | 4² |
| | | carrierparticle-MMC Med | | | | | |
| 3 | 3 | magnetically targetable | 0.12 | 2.30 | 4 | 80 | 16² |
| | | carrier particle-MMC High | | | | | |
| 4 | 3 | magnetically targetable | 0 | 2.37 | 0 | 80 | 16³ |
| | | carrier particle High Control | | | | | |
| 5 | 3 | MMC High Control | 0.11 | 0 | 4 | 0 | 16⁴ |
| 6 | 3 | Vehicle Control | 0 | 0 | 0 | 0 | 16 |

¹The dose in mg/kg was estimated based on the average swine weight for each treatment group.
²The dose solution had a concentration of 0.25 mg/mL of mitomycin and 5.0 mg/mL of magnetically targetable carrier particle drug carrier.
³The dose solution had a concentration of 5.0 mg/mL of magnetically targetable carrier particle drug carrier.
⁴The dose solution had a concentration of 0.25 mg/mL of mitomycin.

The magnetically targetable carrier-MMC particle injectable suspensions from the above examples used in the low, medium, and high dose groups were sonicated and syringe mixed prior to use. Each animal received a single dose of either control or test article at fixed concentrations by pulmonary artery infusion. The low dose magnetically targetable carrier-MMC particle group received 0.25 mg of MMC and 5 mg of magnetically targetable carrier particle in a 1 mL dose volume. The medium dose magnetically targetable carrier-MMC particle group received 1 mg of MMC and 20 mg of magnetically targetable carrier particle in a 4 mL dose volume. The high dose magnetically targetable carrier-MMC particle group received 4 mg of MMC and 80 mg of magnetically targetable carrier particle in a 16 mL dose volume. The magnetically targetable carrier particle control group received 80 mg of magnetically targetable carrier particle in a dose volume of 16 mL and the MMC control group received 4 mg MMC in a dose volume of 16 mL. The vehicle control group received 16 mL of vehicle.

### Administration of Compositions

The animals were fasted overnight prior to surgery. In preparation for the procedure, each animal was pre-anesthetized with ketamine (20 mg/kg im) and xylazine (1.25 mg/kg im). Under general isoflurane anesthesia, percutaneous access into the right common femoral vein was achieved with a 21 gauge single wall needle (Boston Scientific, Natick, Mass.). A 0.018″ nitinol mandril wire (Boston Scientific, Natick, Mass.) was advanced centrally under fluoroscopic guidance and a 4 French coaxial introducer (Boston Scientific, Natick, Mass.) was threaded over the wire. This allowed introduction of a 0.035″ glide wire (Terumo, Elkton, Md.) and subsequent placement of a 5 French Vascular Sheath (Terumo, Elkton, Md.). Animals were administered 5000 IU heparin systemically immediately after sheath placement as prophylaxis against catheter induced thrombosis.

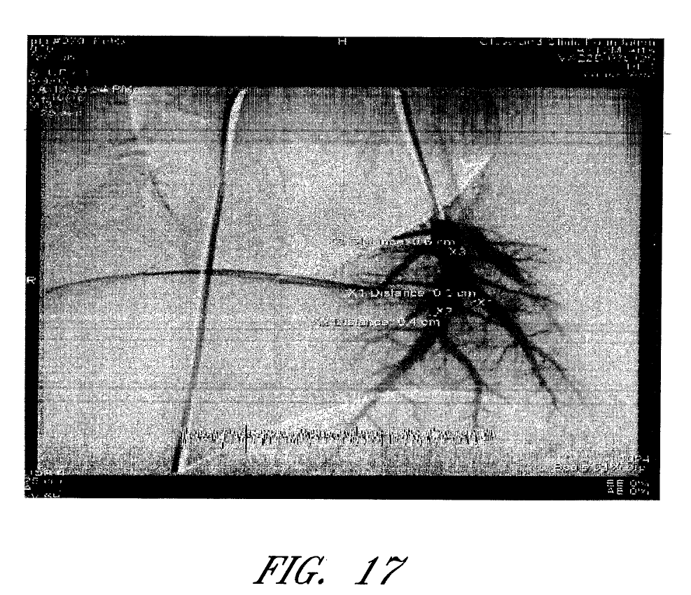

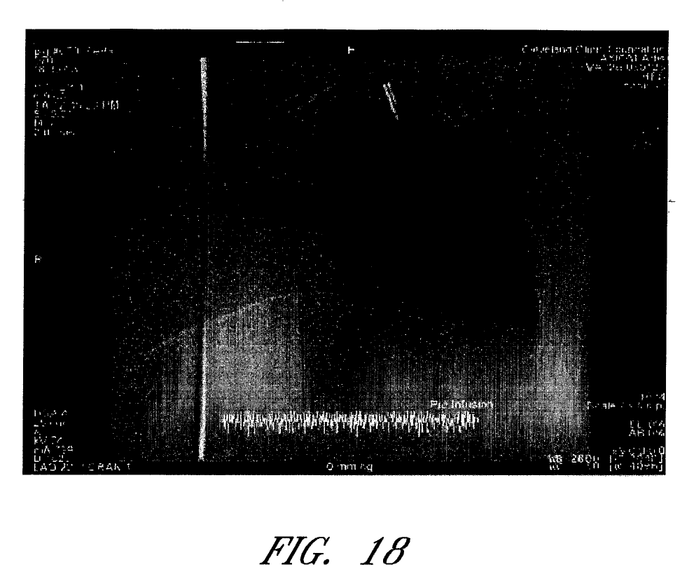

Under fluoroscopy, either a 5 French Cobra 2 angiographic catheter (Cook, Inc, Bloomington, Ind.) or a 5 French Kumpe angled angiographic catheter (Cook, Inc, Bloomington, Ind.) was used for initial access to the right ventricle and the pulmonary outflow tract. An angiogram was performed to delineate the main right and left sided pulmonary arteries. A steerable glide wire was used in combination with the angiographic catheter to engage the desired pulmonary artery. Further, selective angiography was performed to select a branch of the pulmonary artery that provided adequate accessibility to the desired lobe of the lung to which the test article was targeted. An over the guidewire exchange allowed introduction of a straight taper 5 French 100 cm glidecatheter (Boston Scientific, Natick, Mass.) to the more distal pulmonary artery. In all study animals, the branch of the pulmonary artery supplying the left lower posterior lung lobe of each animal was selected. Angiography was then performed to verify catheter placement in the desired branch of the pulmonary artery. Representative angiographic images of catheter placement are shown in FIGS. 17 and 18.

Placement of the magnet, used to localize the materials to the desired tissue, was also determined by angiography. A lateral view allowed for determination of depth by measurement of the distance from the catheter tip to the sternum (10-14 cm). An anterior projection allowed for measurement of the distance across from the catheter tip to the skin on the lateral rib cage (4-7 cm). Magnet placement was determined by identifying the site on the ventral surface of the skin central to the capillary blush and approximately 1-2 cm distal to the catheter tip. The north pole of the 5 kgauss rare-earth magnet housed in a flexible magnet holder was centered on the marked position on the skin surface. The magnetic was kept in position during the entire infusion procedure for Groups 1-4, and for an additional 15 min following the completion of infusions.

A test article was administered as a single infusion to Groups 1 and 2 and as two infusions in all other groups. A 15 minute magnetic retention period separated the two infusions in Groups 3 and 4. The infusion rate was maintained at a flow rate of 2 mL/min. At the end of the 15 minute magnetic retention period, an angiogram was performed to verify arterial patency in the selected lung lobe. Assessment of toxicity following treatment included laboratory tests in which blood samples were collected for evaluation of serum chemistry, hematology and coagulation parameters prior to dosing (Day 0) and post-dosing on Days 1, 3, 7, 14, 21 and 28. Additional blood samples were collected from all animals that received magnetically targetable carrier-MMC particle or MMC alone prior to dosing and for 3 hours following treatment for toxicolinetic analysis. Plasma was collected from the blood samples following centrifugation and MMC levels were measured by HPLC (Quest Pharmaceutical Services, Newark, Del.). A complete necropsy examination was conducted on all animals sacrificed at the end of the study 28 day post-dosing. A full panel of more than 40 tissues and/or organs were collected from each animal and preserved in 10% neutral-buffered foramalin. Tissues were embedded in paraffin and sectioned and stained with hematoxlin and eosin for examination by light microscopy.

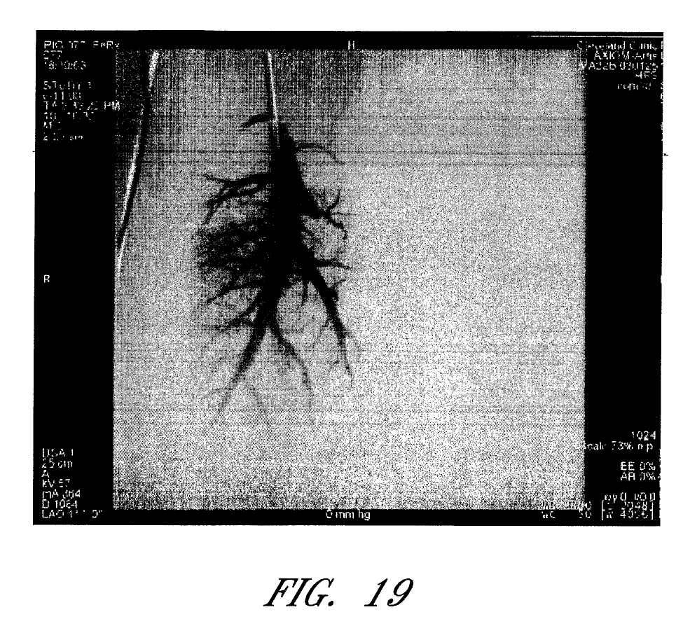

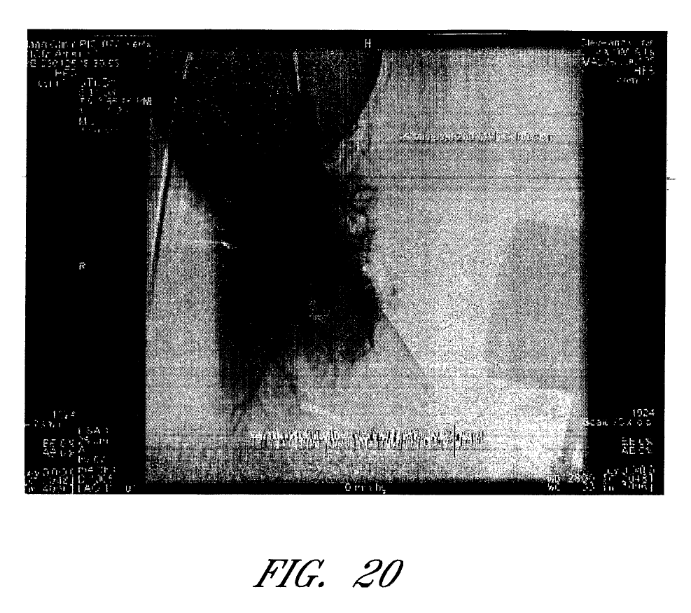

Catheters were positioned similarly in the left lower posterior lung lobe of each animal so that test article was delivered as close to the same site within the lung as possible. Angiography was used following treatment to determine the degree of embolization or potential effects on pulmonary vessels. There was no embolization associated with administration of the low dose of magnetically targetable carrier-MMC particle or MMC or vehicle controls. Distal pruning of pulmonary artery branches were noted in 1 out of 3 animals treated with the medium dose of magnetically targetable carrier-MMC particle and 2 out of the 3 animals treated with magnetically targetable carrier alone (FIGS. 19 and 20). A patchy loss of parenchymal enhancement laterally with adjoining mild peripheral pruning was seen after the second infusion in all animals treated with the high dose of magnetically targetable carrier-MMC particle. All animals appeared to tolerate the treatments well.

There was no mortality and clinical signs were limited to transient diarrhea in two animals from the MMC alone group and two animals from the vehicle control group. Body weight gains were reduced over the course of the study, but they were similar in all treatment groups.

Treatment-related changes in clinical chemistry, hematology and coagulation parameters were minor and transient. There were some differences among groups in alanine aminotransferase, albumin, bicarbonate, creatine kinase, creatinine, potassium, urea nitrate, mean corpuscular hemoglobin concentration, mean corpuscular volume, lymphocytes, neutrophils, platelets, white blood cells and prothrombin time on one or more days that were not consistent and generally not dose-related. There were few changes in hematology parameters either between treatment groups or over time. In general serum chemistry and hematology parameters did not appear to be affected by treatment. A Dunnett's t-test was used to compare each treatment group to the vehicle control group. Table 7 includes those parameters in which there was a significant p-value of <0.05 when individual treatment groups were compared to vehicle control. Prothrombin time demonstrated differences (p<0.05) across multiple time points (i.e., Days 1, 4, 7, 21, and 28), but there was almost no evidence of a dose-related response. Activated partial thromboplastin was not affected by treatment.

TABLE 7
Clinical Pathology Differences: Comparison
of Treatment Groups to Vehicle Control

| | | |
|---|---|---|
| Laboratory | | |
| Measurement | Time Point(s) | Treatment Group(s) |

Clinical Chemistry

| | | |
|---|---|---|
| ALT | Day 28 | High Dose magnetically targetable |
| | | carrier-MMC particle |
| | | magnetically targetable carrier |
| Absolute | Day 21 | High Dose magnetically targetable |
| Active | | carrier-MMC particle |
| Lymphocytes | | |
| Absolute | Days 14, 21 | Med. Dose magnetically targetable |
| Lymphocytes | | carrier-MMC particle |
| Absolute | Day 7 | Med. Dose magnetically targetable |
| Monocytes | | carrier-MMC particle |
| Albumin | Day 7 | Med. Dose magnetically targetable |
| | | carrier-MMC particle |
| | | High Dose magnetically targetable |
| | | carrier-MMC particle |
| Bicarbonate | Day 4 | magnetically targetable carrier |
| | | particle |
| CK | Day 1 | Low Dose magnetically targetable |
| | | carrier-MMC particle |
| Creatinine | Day 28 | High Dose magnetically targetable |
| | | carrier-MMC particle |
| | | magnetically targetable carrier |
| Potassium | Day 4 and 28 | High Dose magnetically targetable |
| | | carrier-MMC particle on Day 4 |
| | | Low dose magnetically targetable |
| | | carrier-MMC particle and High Dose |
| | | magnetically targetable carrier-MMC |
| | | particle on Day 28 |
| Urea nitrate | Days 14, 28 | High Dose magnetically targetable |
| | | carrier-MMC particle on Day 14 |
| | | Med. Dose magnetically targetable |
| | | carrier-MMC particle on Day 28 |

Hematology

| | | |
|---|---|---|
| Lymphocytes % | Day 4 | magnetically targetable carrier |
| | | particle |
| MCHC | Day 21 | All groups |
| MCV | Day 1 | magnetically targetable carrier |
| | | particle |
| Monocytes % | Day 7 | Med. Dose magnetically targetable |
| | | carrier-MMC particle |
| Segmented | Day 4 | magnetically targetable |
| Neutrophils % | | carrier particle |
| Absolute | Day 4 | Low Dose magnetically targetable |
| Segmented | | carrier-MMC particle |
| Neutrophils | | |
| Platelets | Day 1 | High Dose magnetically targetable |
| | | carrier-MMC particle |
| WBC | Day 1 | Med. Dose magnetically targetable |
| | | carrier-MMC particle |

Animals treated with magnetically targetable carrier-MMC particle had less circulating levels of MMC at all time points measured over the 3 hr sampling period than those treated with MMC alone. Calculated pharmacokinetic parameters are shown in Table 8. These data show that whether MMC was administered as magnetically targetable carrier-MMC particle or as MMC itself, the drug was rapidly distributed with a distribution half-life of approximately 8 minutes, followed by an elimination half-life of approximately 65 minutes. Magnetically targetable carrier-MMC particle showed linear pharmacokinetics, meaning that the Area Under the Curve (AUC) increased proportionally with dose and that Total Body Clearance (CL) values did not seem to be dose-dependent. Decreases in the CL and Volume, steady state (Vss) seen with MMC administered as MMC rather than magnetically targetable carrier-MMC particle is consistent with the higher plasma AUC seen when 4 mg MMC was administered rather than 4 mg MMC equivalents as magnetically targetable carrier-MMC particle.

TABLE 8
Calculated pharmacokinetic parameters for MMC in swine

| | AUC | Cₘₐₓ | CL | Vss | αt_{1/2} | β or λ t_{1/2} |
|---|---|---|---|---|---|---|
| Treatment | (ng/ml) × min | ng/ml | ml/min | L | min | min |
| Group 1 magnetically | 208 ± 75 | 3.0 ± 1.1 | 1344 ± 595 | 67.9 ± 29.2 | — | 39.0 ± 2.3 |
| targetable carrier-MMC | | | | | | |
| particle (0.25 mg MMC) | | | | | | |
| Group 2 magnetically | 1433 ± 307 | 40.0 ± 2.0 | 718 ± 143 | 52.6 ± 12.4 | 8.3 ± 1.3 | 65.9 ± 18.6 |
| targetable carrier-MMC | | | | | | |
| particle (1 mg MMC) | | | | | | |
| Group 3 magnetically | 3553 ± 993 | 108.2 ± 34.9 | 1181 ± 295 | 89.8 ± 36.3 | 7.9 ± 2.5 | 68.0 ± 24 |
| targetable carrier-MMC | | | | | | |
| particle (4 mg MMC) | | | | | | |
| Group 5 MMC alone | 6507 ± 568 | 177.8 ± 27.1 | 618 ± 54 | 47.3 ± 13.2 | 8.3 ± 2.4 | 67.0 ± 16.7 |
| (4 mg MMC) | | | | | | |

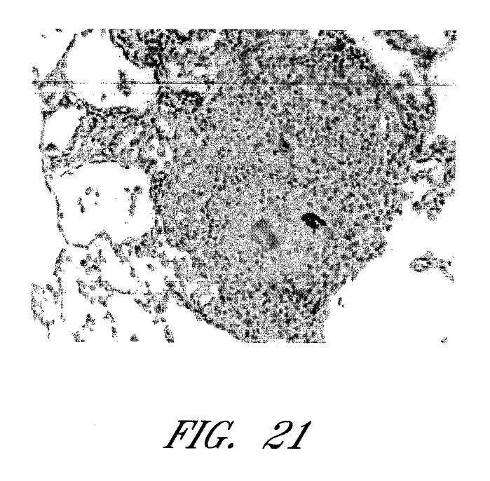

Upon necropsy at the end of the study 28 days post-dosing, gross lesions were observed in seven of the 18 animals and included two animals from the medium dose magnetically targetable carrier-MMC particle group, two animals from the high dose magnetically targetable carrier-MMC particle group, two animals from the magnetically targetable carrier particle alone group and one animal from the vehicle control group. However, the only gross lesion considered related to treatment was the presence of multiple pulmonary granulomas in the targeted lung of one animal (FIG. 21) that was treated with the high dose of magnetically targetable carrier-MMC particle. Many of the granulomas contained magnetically targetable carrier particles at their centers.

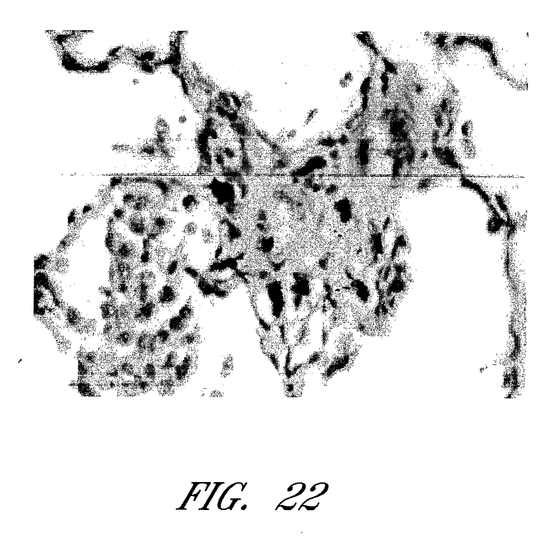

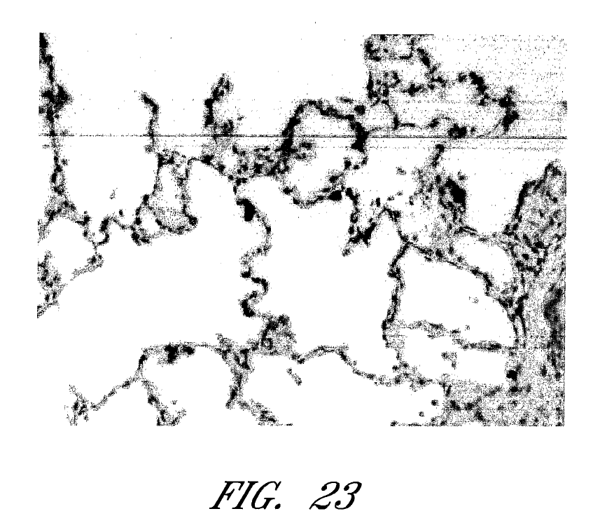

Direct treatment-related microscopic changes were limited to the targeted region of the lung in those groups administered magnetically targetable carrier particle or magnetically targetable carrier-MMC particle. The presence of magnetically targetable carrier particles was confined to the targeted lung lobes of these animals. Magnetically targetable carrier particles were observed in alveolar capillaries of most animals (FIGS. 22 and 23). No magnetically targetable carrier particles were observed in the targeted lung of two animals that received the low dose of magnetically targetable carrier-MMC particle that contained 5 mg magnetically targetable carrier particle. Magnetically targetable carrier particles may not have been detected in these instances because of the small amount of magnetically targetable carrier particles administered, or possibly sampling of the lung did not include the targeted region. The presence of magnetically targetable carrier particles in the targeted lung did not appear to be associated with a pathologic lesion other than the presence of multifocal granulomas noted in one animal treated with the high dose of magnetically targetable carrier-MMC particle. Magnetically targetable carrier particles could be seen at the center of many of the granulomas. The absence of similar lesions in animals treated with the high dose of magnetically targetable carrier particles alone suggest a possibility that the association of MMC with magnetically targetable carrier particles at higher doses may play a role in initiating a foreign body reaction.

No magnetically targetable carrier particles or treatment-related abnormalities were found in the non-targeted lung of any animals. Pre-existing inflammation was prevalent perivascularly within the walls of bronchi and bronchioles, within airway lumina, and within alveoli of both lobes of the lung. The inflammation was multifocal, acute to subacute, and in most cases, minimal to mild. The presence of similar inflammatory processes in the non-targeted lung lobe, as well as the lungs of animals from the control groups, suggest a pre-existing pathologic process unrelated to the test article.

Magnetically targetable carrier particles were not present within any tissues other than targeted lung. Specifically, no magnetically targetable carrier particles were noted within the reticulo-endothelial system. Lesions in non-pulmonary tissues were considered pre-existing and unrelated to administration of the test article. Thymic atrophy was present in some animals receiving magnetically targetable carrier-MMC particle and stomach ulceration was seen in some animals receiving magnetically targetable carrier-MMC particle or MMC, but was not dose-related. The lesions that were seen consisted predominantly of subacute and minimal to mild inflammation in the kidney, urinary bladder, esophagus, tongue, skin, gallbladder, salivary gland, and stomach. Inflammation occurred in control groups at a similar incidence to the treatment groups.

Biologically significant gross and microscopic lesions were limited to the targeted area of lung in one animal receiving the high dose of magnetically targetable carrier-MMC particle containing 80 mg magnetically targetable carrier particle and 4 mg MMC. Magnetically targetable carrier particles were found only in the targeted lung lobe of animals treated with magnetically targetable carrier-MMC particle or magnetically targetable carrier particles demonstrating localization and retention of the drug carrier. Circulating blood levels of MMC were at least half the levels in magnetically targetable carrier-MMC particle treated animals compared to MMC control animals, suggesting that the drug was in contact with the targeted site for a longer period of time in magnetically targetable carrier-MMC particle treatment groups. The no-adverse-effect level was considered to be the magnetically targetable carrier-MMC particle medium dose group containing 20 mg magnetically targetable carrier and 1 mg MMC.

1-34. (canceled)

35. A magnetically targetable carrier composition comprising;
composite particles of activated carbon and iron, wherein said carbon is randomly distributed throughout the particle volume, wherein said particles includes a ratio of weight of iron to activated carbon in the range of from about 95:5 to about 50:50, and wherein said particles include a ratio of weight of mitomycin C to a combined carbon and iron weight in the range of from about 1:100 to about 20:100.

36. The magnetically targetable carrier composition of claim 35, wherein said ratio of weight of mitomycin C to a combined carbon and iron weight is in the range of from about 1:100 to about 10:100.

37. The magnetically targetable carrier composition of claim 36, wherein said ratio of weight of mitomycin C to a combined carbon and iron weight is about 5:100.

38. The magnetically targetable carrier composition of claim 35, wherein the mean size of said particles is between approximately 0.1 μm to approximately 20 μm.

39. The magnetically targetable carrier composition of claim 38, wherein said mean size of said particles is less than 5 μm.

40. The magnetically targetable carrier composition of claim 38, wherein a major dimension of about 95% of particles is between about 0.5 μm and about 5 μm.

41. The magnetically targetable carrier composition of claim 35, wherein said activated carbon is selected from the group consisting of Norit A, B, E, K, KB and chemically modified versions and combinations thereof.

42. The magnetically targetable carrier composition of claim 41, wherein said weight ratio of iron to activated carbon is selected from the group consisting of from about 85:15 to about 60:40, from about 86:14 to 64:36 and about 65%:35%.

43. The magnetically targetable carrier composition of claim 35, wherein said iron contains less than about 5% iron oxide.

44. The magnetically targetable carrier composition of claim 41, wherein the mean diameter of the particles is between about 0.6 and about 3.5 microns.

45. The magnetically targetable carrier composition of claim 35, wherein said particles have an adsorption capacity for an additional biologically active substance of up to 20% of the mass of the particle.

46. The magnetically targetable carrier composition of claim 45, wherein said particles have a therapeutically effective amount of said additional biologically active substance adsorbed thereon, said biologically active substance being different from mitomycin C, and said carbon is activated carbon.

47. The magnetically targetable carrier composition of claim 45, wherein said additional biologically active substance is selected from the group consisting of a drug, a radioactive substance, genetic material, antibiotics, antifungals, an additional antineoplastic agents and combinations thereof.

48. A formulation for a magnetically targetable carrier composition and mitomycin C, wherein said formulation comprises:
a) composite particles of activated carbon and iron, wherein said carbon is randomly distributed throughout the particle volume, wherein said particles includes a ratio of weight of iron to activated carbon in the range of from about 95:5 to about 50:50, and wherein said particles include a ratio of weight of mitomycin C to a combined carbon and iron weight in the range of from about 1:100 to about 20:100; and
b) a delivery vehicle.

49. The formulation of claim 48, wherein the concentration of mitomycin C is selected from the group consisting of between about 0.5 to about 1 mg/mL and about 0.75 mg/mL before adsorbtion to the magnetically targetable carrier.

50. The formulation of claim 48, further comprising an excipient.

51. The formulation of claim 50, wherein said excipient is mannitol.

52. The formulation of claim 48, wherein said delivery vehicle comprises:
a) a sugar;
b) a polymer; and
c) a solvent.

53. The formulation of claim 52, wherein said sugar is mannitol.

54. The formulation of claim 52, wherein said polymer is carboxymethylcellulose.

55. The formulation of claim 54, wherein said delivery vehicle comprises a solution with a viscosity of about 14±2 cP when measured at 4 OC and 60 rpm in a Brookfield viscometer.
